SEQUENCE LISTING (5 sequences)

SEQ ID NO: 1 - protein, 295 aa, Homo sapiens
METPAWPRVPRPETAVARTLLLGWVFAQVAGASGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFREIFYIIGAVVFVVIILVIILAISLHKCRKAGVGQSWKENSPLNVS

SEQ ID NO: 2 - protein, 286 aa, Homo sapiens
MERLRLMFLLILMVVLFHVFVDASGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFREIFYIIGAVVFVVIILVIILAISLHKCRKAGVGQSWKENSPLNVS

SEQ ID NO: 3 - DNA, 10808 bp, Artificial Sequence
  Description of Artificial Sequence Synthetic nucleotide construct
ggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgtcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactcatgtttgacagcttatcatcggatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggccggccagggatctccggactagtttatgaaacattcagtggggagttctccttccagctctgccccactcctgcctttctacacttgtgtagagatatagccaggatgatgacaaggatgatgaccacaaataccacagctccaatgatgtagaatatttctctgaattcccctttctcctggcccatacactctaccgggctgtctgtactcttccggttaactgttcgggagggaatcactgcttgaacactgaaacagtagttttctcctttatccacatcaatcaaaaactcattagtgtttgttttggctgttttctttcctgaacttgaagatttccaataataaagtgtataaattaagtccttgccaaaaacatcccggaggcttaggaaagtgttgttccttctgactaaagtccgttcatcttctacggtcacattcacttttgttcccacctgttcaaaactctgaattgttggctgtccgaggtttgtctccaggtaaggtgtgaactctggggagttctcatacagaggctccccagcagaaccggtgctctccacattccctgccgggtaggagaagacccgtgccaagtacgtctgcttcacatccttcacaatctcgtcggtgaggtcacactctgtgtctgttgtgtaaaagcatttgcttttccaatctcctgacttagtgcttatttgaacagtgtagacttgattgacgggtttgggttcccactccaaaattgtcttgaaattagttgatttccaagttaaattatatgctgccacagtatttgtagtgcctgatgcatcaacaaacacatgaaacaacactaccattagtattaaaagaaacatgagcctgagtctttccatggggggtgtacagtcgactgcaggttctagagtcgatttggtgtatcgagattggttatgaaattcagatgctagtgtaatgtattggtaatttgggaagatataataggaagcaaggctatttatccatttctgaaaaggcgaaatggcgtcaccgcgagcgtcacgcgcattccgttcttgctgtaaagcgttgtttggtacacttttgactagcgaggcttggcgtgtcagcgtatctattcaaaagtcgttaatggctgcggatcaagaaaaagttggaatagaaacagaatacccgcgaaattcaggcccggttgccatgtcctacacgccgaaataaacgaccaaattagtagaaaaataaaaactgactcggatacttacgtcacgtcttgcgcactgatttgaaaaatctccctcgagaaagagatcaatgttgagctgcttcaaaagcaatgggattgacctgcagcccaagcttggaattcgggatccctgaaagcgacgttggatgttaacatctacaaattgccttttcttatcgaccatgtacgtaagcgcttacgtttttggtggacccttgaggaaactggtagctgttgtgggcctgtggtctcaagatggatcattaatttccaccttcacctacgatggggggatcgcaccggtgagtaatattgtacggctaagagcgaatttggcctgtaggatcccgaattccaagcttggaattcgggatccctgaaagcgacgttggatgttaacatctacaaattgccttttcttatcgaccatgtacgtaagcgcttacgtttttggtggacccttgaggaaactggtagctgttgtgggcctgtggtctcaagatggatcattaatttccaccttcacctacgatggggggcatcgcaccggtgagtaatattgtacggctaagagcgaatttggcctgtaggatcccgaattccaagcttggaattcgggatccctgaaagcgacgttggatgttaacatctacaaattgccttttcttatcgaccatgtacgtaagcgcttacgtttttggtggacccttgaggaaactggtagctgttgtgggcctgtggtctcaagatggatcattaatttccaccttcacctacgatggggggcatcgcaccggtgagtaatattgtacggctaagagcgaatttggcctgtaggatcccgaattccaagcttatcgataccgtcgaacgcggggtaccgagctcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagctttcgcagatctgtcgatcgaccatggggattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcgcaggatgatctggacgaagagcatcaggggctcgcgccagccgaactgttcgccaggctcaaggcgcgcatgcccgacggcgaggatctcgtcgtgacacatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcggatcgatcctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgc

SEQ ID NO: 4 - protein, 263 aa, Homo sapiens
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFREIFYIIGAVVFVVIILVIILAISLHKCRKAGVGQSWKENSPLNVS

SEQ ID NO: 5 - protein, 32 aa, Homo sapiens
METPAWPRVPRPETAVARTLLLGWVFAQVAGA

(19) United States
(12) Patent Application Publication
(10) Pub. No.: US 2007/0292490 A1
(43) Pub. Date: Dec. 20, 2007

(54) Production of tissue factor in plants

(76) Inventors: Valentin Negrouk, Weston, FL (US); Hing C. Wong, Weston, FL (US); Dean Taylor, Weston, FL (US); Kai-ping Han, Miramar, FL (US)

Correspondence Address: EDWARDS ANGELL PALMER & DODGE LLP, P.O. BOX 55874, BOSTON, MA, 02205, US

(21) Appl. No.: 11/165,590

(22) Filed: Jun. 22, 2005

Related U.S. Application Data
(60) Provisional application No. 60/583,187, filed on Jun. 25, 2004.

(51) Int. Cl. A61K 38/36; A01H 5/00; A61P 7/02; C12N 15/82; C12P 21/00; C07K 14/415; A61F 13/00

(52) U.S. Cl. 424/446; 435/320.1; 435/69.1; 514/12; 530/370; 800/288; 800/298; 800/305; 800/312; 800/313; 800/314; 800/315; 800/316; 800/317.1; 800/317.2; 800/317.3; 800/317.4; 800/318; 800/320.1; 800/320.2; 800/320.3; 800/322

(57) ABSTRACT

The instant invention provides transgenic plants that express mammalian, e.g., human, recombinant tissue factor (rhTF) as well as methods for making rhTF. The invention further provides rhTF or functional fragment thereof that are obtained from a transgenic plant. The invention also provides methods of treating a subject using the rhTF or a fragment thereof.

### CROSS-REFERENCE TO RELATED APPLICATION

The present application is a continuation-in-part of U.S. Ser. No. 60/583,187 as filed on Jun. 25, 2004 and claims priority to U.S. Ser. No. 60/583,187. The disclosure of said U.S. Ser. No. 60/583,187 is incorporated herein by reference.

### FIELD OF THE INVENTION

The invention generally relates to the production and use of plant-derived tissue factor as well as functional fragments of that protein. Tissue factor is a key component of blood coagulation cascade. The invention has a wide spectrum of useful applications including providing highly active tissue factor for use in the prevention or treatment of bleeding and wound healing.

### BACKGROUND

There is general acknowledgement that an important step in wound treatment is to reduce or eliminate bleeding. That step usually requires blood coagulation not only to stem the loss of blood but to initiate wound closure and healing. More generally, blood coagulation is thought to help restore homeostasis and promote faster and more efficient wound healing. See generally L. Stryer, Biochemistry, 3rd Ed, W.H. Freeman Co., New York; A. G. Gilman et al., The Pharmacological Basis of Therapeutics, 9th Edition, McGraw Hill Inc., New York, pp. 1341-1359; and Mann, K. G. et al. (1992) Semin. Hematol. 29: 213.

Biological pathways that promote blood coagulation have been reported. These include an intrinsic and an extrinsic pathway. See S. I., Rapaport and L. V. M. Rao (1995) Throm. Haemost. 74: 7; and Stryer, L. supra and references cited therein.

The extrinsic pathway of blood coagulation is thought to be triggered by exposure to a blood coagulation factor called Tissue Factor (TF). More specifically, the extrinsic (TF) pathway is thought to start when blood comes in contact with TF. Exposure of flowing blood to TF initiates clotting. In the presence of calcium, TF and FVIIa form a complex (TF-FVIIa) which is thought to catalyze activation of other “downstream” blood factors FX and FIX. FXa and Factor Va form the “prothrombinase” complex” which is believed to convert prothrombin to thrombin. Thrombin helps form the blood clot. See, e.g., Davie, E. W. et al. (1991) Biochem. 30: 10363; Nemerson, Y. (1988) Blood 71: 1, Rand, M. D. et al. (1996) Blood 88: 1; and Monroe, D. M. et al. (1994) Brit. J of Haemot. 88: 364.

The structure and function of tissue factor have been disclosed. See, e.g., U.S. Pat. Nos. 5,110,730; 5,298,599; and 5,622,931. In particular, the nucleic acid and amino acid sequence of human tissue factor has been reported. See GenBank Accession Nos. J02846 (gene sequence) and BC011029 (cDNA, mRNA and protein sequences). It is generally believed that TF requires an association with certain membrane components, typically phospholipids to achieve optimal activity. See Williams Hematology, 5^{th} Ed. (Beutler, E. et al. Eds.) McGraw-Hill, Inc., Health Professions Div., New York.

Methods for determining how quickly blood clots are known. These methods are useful, for instance, for identifying blood factor deficiencies in patients. One standard method (called the prothrombin time (PT) test) uses calcified blood plasma as a starting reagent. Subsequently, the plasma is combined with thromboplastin or TF. Clotting times are then measured. According to a standard method, clot reaction can be initiated by addition of lipidated recombinant human TF (rhTF) in the presence of Ca⁺⁺. An automated coagulation timer can be used to determine the clot time. See e.g., Miletich, J P (1995) in Prothrombin Time (Chapter L33) Williams Hematology, 5^{th} Ed., supra, pages L82-L84.

Although some microorganisms have been used to make rhTF, it is known that such material is not always useful. For instance, there is general recognition that optimal blood clotting activity is achieved with purified TF or rhTF protein preparations are combined with certain phospholipids in a process referred to “relipidation” of the rhTF. This extra processing step adds to the cost of making and using rhTF in many settings. Consistent lipidation of TF is believed to be a critical factor to the success of many in vitro blood clotting assays.

There have been efforts to use plants to make certain heterologous proteins. See e.g., PCT/US02/23624 to Bascomb, N. et al.; PCT/US02/17927 to Hall, G. et al; Daniell, H et al. (2001) Trends in Plant Science, Vol. 6: 219; and references cited therein.

In particular, certain plants have been reported to be an attractive alternative to microbial and animal cell based protein production systems. There are many reports concerning the benefits of producing proteins in plants. These include straightforward purification procedures and the ability to “farm” the protein on a large scale using traditional agronomic practices.

Notwithstanding these benefits, it is not always clear whether all heterologous proteins can be made in plants efficiently. In particular, it is not certain whether plants have the capacity to fold and modify all such proteins following translation such that the protein is soluble and biologically functional. Preferred codon usage necessary to achieve reasonable levels of expression is not always known for many such proteins. Plant membrane components that may impact protein production or function are usually not completely understood. In some cases, plants may suppress the production of certain heterologous proteins by a process referred to as silencing.

Notwithstanding these difficulties, it would be desirable to have transgenic plants that can express tissue factor. It would be further desirable to have plant-derived recombinant tissue factor (rhTF) in which the need for relipidation has been minimized and preferably eliminated. It would also be desirable to have vectors for making the plants as well as compositions and methods that use the plant-derived rhTF to prevent or treat wounds in a patient.

### SUMMARY OF THE INVENTION

The invention generally relates to transgenic plants that express tissue factor (TF) as well as methods for making and using the TF. More particularly, the invention relates to plant-derived TF compositions that assist blood clotting without the need to relipidate the protein. The invention has a wide spectrum of useful applications including use in diagnostic assays and as an agent for promoting wound healing.

We have found that it is possible to express TF in plants and to obtain functional TF protein that requires minimal if any relipidation. That is, we have discovered that plants can supply cellular components that help promote TF function. Without wishing to be bound to theory, it is believed that plant membranes can provide component molecules that can substitute for those with which TF naturally associates (or associates synthetically). This observation is surprising in that there has not been universal acknowledgment that plants can express functional TF that requires minimal if any lipidation to achieve significant blood clotting activity.

These and other surprising features of the invention provide substantial advantages. For example, and in accord with the present invention, it is now possible to express functional TF in plants in a fashion that is more economical than prior methods of using fermentation or bioreactor systems to express TF in microbes. In addition, and in accord with methods provided by the invention, it is now possible to “farm” functional TF on a relatively large scale, i.e., to utilize standard technologies for transforming, harvesting, and processing transgenic plants to make the protein. Moreover, the TF obtained from such methods requires minimal if any relipidation to make functional protein. This invention provides several specific advantages including lowering the cost of obtaining functional TF and providing for TF that has more uniform activity from batch to batch. These advantages, in turn, will help provide better and more efficient treatment to prevent blood loss or promote wound healing and further, for use in existing diagnostic assays such as those intended to monitor blood clotting in vitro.

Accordingly, and in one aspect, the invention provides for a recombinant tissue factor protein or a functional fragment thereof that is obtained from a transgenic plant that expresses the protein or a functional fragment thereof. Preferably, the tissue factor or fragment expressed by the plant is mammalian, such as a rodent or primate. More preferred is a recombinant human tissue factor protein manufactured by the transgenic plant or functional fragment of that protein. Typically preferred recombinant tissue factor protein or fragment regardless of source does not require any significant relipidation to achieve useful activity in a standard prothrombin time (PT) assay. Examples of such PT assays are described below.

More particular recombinant mammalian tissue factor or functional fragment proteins in accord with the invention include an operatively linked plant- or mammal-derived signal peptide.

Other particular recombinant mammalian tissue factor and functional fragments provided herewith include those proteins that include at least one plant-specific glycan. Preferably, that glycan is covalently linked to the protein preferably through one or more intervening carbohydrate groups. Examples of such plant-specific glycans are an α(1,3) fucose group and a β(1,2) xylose group. Still other plant-derived recombinant mammalian tissue factor proteins and functional fragments thereof do not usually include carbohydrates that are indicative of expression in animal cells e.g., one or more of a terminal galactose or sialic-acid group. By “terminal” is meant that the animal specific carbohydrate is typically positioned at or near the end of the glycan structure bound to the protein.

Such recombinant mammalian tissue factor proteins or fragments can be provided in the form of a crude plant extract or in a more purified form as needed to suit a particular invention objective. Thus in one embodiment, the protein or fragment is provided as a substantially pure preparation.

As discussed, such recombinant mammalian tissue factor proteins and fragments provided by the invention do not require significant relipidation to achieve optimal biological activity in a wide variety of assays including those that monitor blood clotting such as a PT test.

The invention further provides for a variety of suitable transgenic plants that include transformed plant cells that express the recombinant mammalian tissue factor or functional fragment thereof. Typically transformed plant cells included within the plant comprise at least one vector that expresses the protein as disclosed herein. Nearly any appropriate monocot or dicot can be transformed by the methods of the invention such as those representative plants described below. Also contemplated are progeny plants, cuttings (sometimes called “slips”), cultivars, or a seed obtained from the tansgenic plant. Preferably, such a progeny plant, cutting, slip, cultivar, or seed includes the vector expressing the mammalian tissue factor or fragment.

Such transgenic plants according to the invention are generally prepared by administering one or more suitable vectors that encode tissue factor or a functional fragment thereof. Thus in another aspect, the invention provides an expression vector that includes in one embodiment: (a) a first promoter operable in a plant; (b) an expressible nucleic acid sequence encoding a mammalian tissue factor protein or functional fragment thereof, wherein the nucleic acid sequence is operatively associated with the promoter; and (c) a termination sequence operatively associated with the nucleic acid sequence. Methods for making and using such vectors are described below.

Further provided by the invention is a crude extract of the transgenic plant that includes the recombinant tissue factor (rhTF) or a functional fragment thereof. More particular extracts include the protein or fragment expressed by the plant suspended in an aqueous fraction. That fraction can be made by one or a combination of standard extraction or purification strategies such as those mentioned below. For most invention embodiments, the crude extract is readily prepared by partially or completely disrupting cells of the plant (or a portion thereof such as the leaves, flowers, etc.) in the presence of an aqueous solution and removing any insoluble components by low speed centrifugation, gravity and the like to make the crude extract.

The crude extract prepared in accord with the invention provides distinct benefits. For instance, it includes functional rhTF that can be used in a variety of applications without the need for lipidation or further purification. That is, the crude extract can be used as a “stand alone” composition (coagulant) or it can be combined with other compositions (e.g., pharmaceutically acceptable vehicles, additives, excipients and the like) to suit an intended use. As will be apparent, the crude extract can be prepared in relatively large quantities, thereby assisting its widespread use in which substantial amounts of rhTF are needed in which the preparation must have essentially consistent biological activity.

Accordingly, and in another aspect, the invention features a coagulant or composition that includes such an agent that comprises the recombinant tissue factor or functional fragment thereof produced by the transgenic plant.

In another aspect, the invention provides for a method of making the transgenic plant disclosed herein. In one embodiment, the method includes at least one and preferably all of the following steps:
i) administering to a suitable plant or plant tissue at least one expression vector comprising (a) a first promoter operable in a plant, (b) an expressible nucleic acid sequence encoding a mammalian tissue factor protein or functional fragment thereof, wherein the nucleic acid sequence is operatively associated with the promoter; and (c) a termination sequence operatively associated with the nucleic acid sequence; and
ii) incubating the plant or plant tissue under conditions conducive to expressing the mammalian tissue factor protein or functional fragment thereof, thereby making the transgenic plant. This invention embodiment is well-suited for a variety of transformation applications including those involving conventional particle bombardment, whisker transformation, electroporation, and protoplast transformation.

If desired, a more particular strategy for making the transgenic plants can be followed as generally provided by these steps:
i) administering to a suitable Agrobacterium strain at least one replicable (in Agrobacterium) expression vector comprising (a) a first promoter operable in a plant, (b) an expressible nucleic acid sequence encoding a mammalian tissue factor protein or functional fragment thereof, wherein the nucleic acid sequence is operatively associated with the promoter; and (c) a termination sequence operatively associated with the nucleic acid sequence; and
ii) incubating the bacterium with a suitable plant or plant tissue under conditions conducive to transforming the plant or plant tissue with the vector to make the transgenic plant. As will be appreciated, this embodiment of the invention is well suited for applications in which use of Agrobacterium-mediated transformation is desired.

Further provided by the invention are pharmaceutically useful forms of the invention suitable for the uses described herein. Included are aqueous suspensions, lyophilized powders, dry powder or liquid aerosol sprays, topical applications such as liquids or creams. Also included are forms in which the rhTF is provided in the form of a solid support, typically an absorbent (or adsorbent) that includes the rhTF or functional fragments disclosed herein. In this invention aspect, the absorbent (or adsorbent) holds the protein for use in embodiments in which more focused and/or prolonged application of the rhTF or fragment is needed. Such a solid support can include an adhesive support adapted to apply the absorbent (or adsorbent). Also envisioned is a wrapper container that includes the adhesive support and is preferably essentially impervious to water, air, and contaminants.

It is an object of the present invention to provide crude plant extracts and substantially purified preparations thereof that can deliver rhTF or a functional fragment thereof to a subject in need of blood clotting. That is, it has been found that the rhTF can be used as a coagulant without significant artificial lipidation. In contrast, most commercial sources of rhTF are produced in a way that requires substantial lipidation to achieve good activity.

Accordingly, it is a further object of the invention to provide a method for promoting wound healing in a subject. In one embodiment, the method includes contacting that subject with a therapeutically effective amount of the coagulant. In a further invention object, there is provided a method for promoting angiogenesis in a subject. Preferably, the method includes contacting the subject with a therapeutically effective amount of the coagulant disclosed herein. It is another invention object to provide a method for promoting vascular remodeling in a subject. In one embodiment, the method includes contacting the subject with a therapeutically effective amount of the coagulant described herein.

In the foregoing invention methods, the subject is preferably a mammal and usually a human patient in need of such treatment. Although these invention methods will find use in a variety of settings, preferred practice will often involve situations in which rapid cessation of blood flow is useful. Such situations include those often associated with a medical, public health, or military emergency such as those associated with a hospital (ER) room, disaster area, or battle field and can also include veterinary applications.

Other aspects and advantages of the invention are described, infra.

### BRIEF DESCRIPTION OF THE DRAWINGS

FIGS. 1A-B are drawings showing the amino acid sequence of human tissue factor (bold) with corresponding signal peptide (underlined) preferred vector (1A). Also shown as FIG. 1B is the sequence of human tissue factor (bold) with tomato subtilase signal peptide (underlined). Putative glycosylation sites are shown (double underline).

FIG. 2 is a drawing showing a physical map of a preferred plant transformation vector (pSUNP12).

FIG. 3 is a photograph of a NuPAGE Bis-tris gel (12%) of purified plant rhTF. Lane 1—molecular weight marker; lane 2—purified lettuce full length (263 aa) rhTF; lane 3—purified E. coli 243aa rhTF.

FIG. 4 is a graph showing results of a PT assay of rhTF₂₄₃ (E. coli derived, purified and relipidated), lettuce-derived rhTF (purified and relipidated) and crude extract of lettuce-derived rhTF at different doses.

FIG. 5 is a graph with results of a PT assay showing the effect of anti-TF antibody, Sunol-CH₃₆, on E. coli rhTF243 and lettuce-derived rhTF.

FIG. 6A-E are photographs of wounds after treatment with water (dsH2O; 6A), PBS (6B), E. coli TF (243; 6C), purified plant TF (6D), and plant crude extract TF (6E).

FIG. 7A-F is a drawing showing the complete nucleotide sequence of the vector represented schematically in FIG. 2.

### DETAILED DESCRIPTION

As discussed, the invention relates to transgenic plants that express mammalian and preferably human recombinant tissue factor (rhTF) as well as methods for making and using that rhTF. Such plant-derived protein has been found to greatly assist blood clotting without the need to perform any relipidation steps. The invention has a wide spectrum of useful applications including use as a coagulant, for promoting wound healing and related functions such as angiogenesis and in diagnostic assays (e.g., PT, aPTT assays).

As also discussed, the invention provides, in one aspect, rhTF or a functional fragment thereof that is obtained from a transgenic plant that expresses the protein or fragment. By the phrase “tissue factor protein” is meant a mammalian tissue factor protein, preferably mature human tissue factor protein as represented by the amino acid sequence shown below in Table IA. The information can also be found at Genbank Accession No. BC011029. See the National Center for Biotechnology Information (NCBI)-Genetic Sequence Data Bank (Genbank), National Library of Medicine, 38A, 8N05, Rockville Pike, Bethesda, Md. 20894. See generally Benson, D. A. et al. (1997) Nucl. Acids. Res. 25:1 for a description of Genbank.

TABLE I

A. Mature Human Tissue Factor Sequence (1-263)
(SEQ ID NO:_{——}):
SGTTNTVAAYNLTWKSTNFKTILEWEPKPVNQVYTVQISTKSGDWKSKCF
YTTDTECDLTDEIVKDVKQTYLARVFSYPAGNVESTGSAGEPLYENSPEF
TPYLETNLGQPTIQSFEQVGTKVNVTVEDERTLVRRNNTFLSLRDVFGKD
LIYTLYYWKSSSSGKKTAKTNTNEFLIDVDKGENYCFSVQAVIPSRTVNR
KSTDSPVECMGQEKGEFREIFYIIGAVVFVVIILVIILASISLHKCRKAG
VGQSWKENSPLNVS
B. Human Tissue Factor Signal Peptide Sequence
(SEQ ID NO:_{——})
METPAWPRVPRPETAVARTLLLGWVFAQVAGA

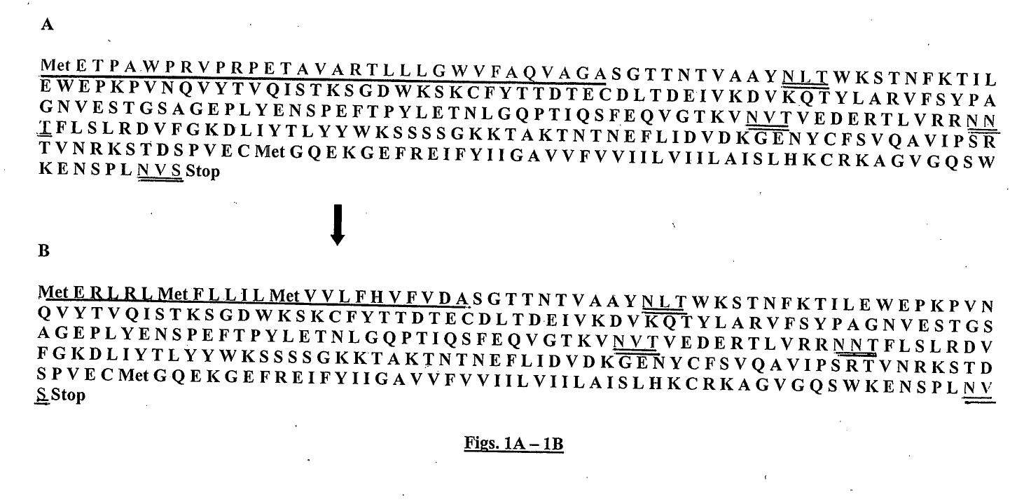

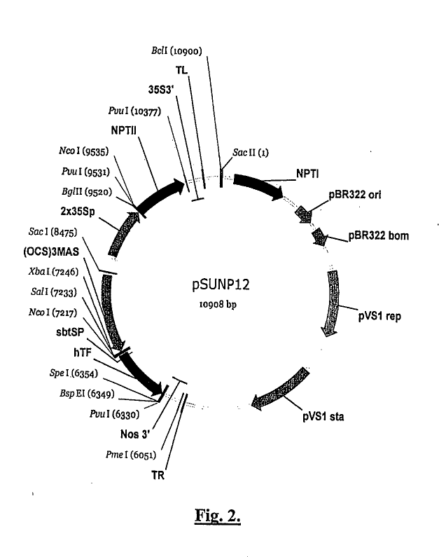

See also FIG. 1A (showing the amino acid sequence of immature human tissue factor (SEQ ID NO.) i.e., the mature sequence (Table IA) with the signal peptide (Table IB). References herein to an amino acid position of the human tissue factor protein refer to the mature protein shown above as Table IA unless otherwise specified.

By the phrase “recombinant” is meant that the techniques used for making the plant-derived tissue factor protein or fragments including those generally associated with making recombinant nucleic acid methods as well as plant culture and transformation (e.g., electroporation, lipofection). Generally, enzymatic reactions and purification steps are performed according to a manufacturer's specifications or in accordance with accepted protocols. The techniques and procedures are generally performed according to conventional methods in the art and various general references. See generally, Sambrook et al., Molecular Cloning: A Laboratory Manual, 2nd. edition (1989) Cold Spring Harbor Laboratory Press, Cold Spring Harbor, N.Y.

For more plant-specific recombinant methods, see Roger P. et al. (2000) Plant Mol. Biol. 42:819; Gartland K. M. A. and Davey M. R. (1995) Agrobacterium Protocols IN Methods in Molecular-44, Humana Press, Totowa, N.J.; Hellens, R. et al. (2000) Trends in Plant Science, 5:10: 446; and Christou, P. (1996) Trends Plant Sci. 1: 423, for instance as well as references cited therein.

Also meant by the phrase “human tissue factor” including “rhTF” are allelic variants of the mature human sequence shown in Table I (SEQ ID NO. ______) as well as sequences that are at least about 70% identical thereto at the protein level, preferably at least about 80% identical, more preferably at least about 90% identical, and more preferably at least about 95%, about 99% or 100% identical. Methods for determining amino acid sequence identity are generally known in the field and include determinations made by using WU-BLAST software (Washington University BLAST) version 2.0. That program is reported to build on WU-BLAST version 1.4, which in turn is based on the public domain NCBI-BLAST version 1.4 (Altschul and Gish, (1996), Doolittle ed., Methods in Enzymology 266:460-480; Altschul et al., (1990), Journal of Molecular Biology 215:403-410; Gish and States, (1993), Nature Genetics 3:266-272; and Karlin and Altschul, (1993), Proc. Natl. Acad. Sci. USA 90:5873-5877). WU-BLAST version 2.0 executable programs for several UNIX platforms can be downloaded, for instance, from the Washington University BLAST website.

Examples of human tissue factor sequences that are less than 100% identical to the specified sequence are those having gaps (consecutive and/or non-consecutive gaps of less than about 5 amino acids such as 1 or 2 amino acids) as well as those sequences featuring conservative amino acid substitutions (e.g., alanine substituted for leucine). Also envisioned are human tissue factor sequences having about 10 amino acid additions or less added to one or both of the N- or C-terminii.

By “functional fragment” of human tissue factor is meant a fragment of a full-length human tissue factor sequence, particularly the mature sequence shown in Table I (SEQ ID NO. ______), including fragments containing the first 243 or 219 amino acids of the mature rhTF sequence. Such functional fragments of that sequence will demonstrate good blood clotting activity in a conventional prothrombin time (PT) or aPTT test. More specific functional fragments will have at least 70% of the blood clotting activity of the mature human tissue factor sequence shown in Table I (SEQ ID NO. ______) in a standard prothrombin time (PT) assay. More preferred human tissue factor sequences and fragments will have at least 80% of the blood clotting activity of the sequence, more preferably at least about 90%, about 95%, up to about 100% of that activity. Examples of suitable fragments are generally known in the field and have been disclosed, for instance, in U.S. Pat. Nos. 6,001,978; 5,110,730; and 5,110,730. By the phrase “rhTF” is meant recombinant human tissue factor.

An especially preferred human tissue factor protein sequence is shown (bound to a plant-derived signal peptide) in FIG. 1B (SEQ ID NO. ______) at amino acid positions 1 to 263. See also Examples 1-3 below, for a preferred method of making that sequence. Also preferred is human tissue factor protein sequence from amino acid positions 1 to 243, fused to a plant-derived signal peptide.

In mammalian cells, TF is found as a transmembrane protein on the surface of mammalian cells that activates the extrinsic pathway for blood coagulation. It functions as a receptor that binds Factor VIIa. The TF-FVIIa complex functions to bind and activate Factor IX or X via limited proteolysis. The conversion of Factor IX or Factor X is considerably more efficient when the TF-FVIIa complex is associated with a membrane (Kalafatis, et al. (1997) Critical Reviews in Eukaryotic Gene Expression 7:241-280). In the case where TF protein is purified from a mammalian source or as a recombinant protein (produced in a heterologous host such as E. Coli and purified), the TF protein must be relipidated to provide an artificial membrane for optimal functioning. The purification of the TF protein is an expensive process and the relipidation is expensive due to the cost of the artificial lipids used and the additional processing. A process for producing functionally active TF without the need to purify or relipidate the TF protein would be commercially important.

The targeting of the TF protein to the cell membrane is provided by a leader or signal sequence that is removed by proteolysis from the mature transmembrane protein. In the case of plants producing recombinant proteins, it has been shown that the mammalian signal sequence can function to target the protein to the membrane (Schillberg, et al. (1999) Transgenic Research 8:255-263), but plant derived signal sequences work better. Such plant signal sequences can include the signal sequence for the tomato subtilase (Janzik, et al. (2000) J. Biol. Chem. 275:5193-5199), the signal sequence for the rice alpha-amylase (McCormick, et al. (1999) PNAS 96:703-708), and the signal sequence for the Nicotiana calreticulin (Komarnytsky, et al. (2000) Plant Physiology 124:927-933).

As discussed, the invention features a recombinant mammalian tissue factor or functional fragments thereof that include an operatively linked and plant-derived signal peptide. Preferably, the signal sequence is derived from any suitable plant such as tomato, rice, tobacco, mustard, cotton maize or alfalfa.

During the translocation of the TF to the membrane, the TF protein typically undergoes post-translational modification, such as glycosylation. The full-length tissue factor protein has four sites for potential N-linked glycosylation and the 243 amino acid form of TF has three sites for potential N-linked glycosylation. The nature of the post-translational glycosylation by plants and mammalian cells are similar in that they contain N-acetyl glucosamine, fucose and mannose, although the nature of the linkages differ. But they differ in that plant glycosylation includes xylose which is not found in mammalian cells, and mammalian glycosylation can have terminal galactose and sialic acid residues not found in plant glycan structures (Ma, et al. (2003) Nature Reviews/Genetics 4:794-805).

Plant-specific glycans are known and have been reported. Generally, the glycans are added post-translationally to form a long-chain glycan structure. By “long-chain” is meant less then about 10 carbohydrate units (e.g., mannose, xylose, sialic acid, etc.) as a unbranched or preferably a branched glycan structure. More specifically, each of the glycans are added to one or more suitable glycosylation sites on a protein as the protein passes through the secretory pathway. Most plant-expressed proteins including those disclosed herein will lack at least one of and preferably both of terminal galactose and sialic acid residues that are usually found in mammals. By “terminal” is meant that the carbohydrates would be present (in mammals) at the end of the long-chain glycan structure. However unlike mammals, the plant expressed proteins will include at least one of and preferably both of a α(1,3) fucose and a β(1,2) xylose group. See generally Warner, T. G. (2000) in Carbohydrates in Chemistry and Biol. (eds. Ernst, B. et al.) pp. 1043-1064 (Wiley, NY).

Accordingly, and in one embodiment, the invention provides a recombinant mammalian tissue factor protein that includes at least one plant-specific glycan which is preferably at least one of the α(1,3) fucose and a β(1,2) xylose group. In one embodiment, the α(1,3) fucose will be fused (covalently linked) to a N-acetylglucosamine (GlcNAc) group which GlcNAc group can, in one embodiment, be bound directly to a protein glycosylation site. Depending, for example, on the plant selected, the GlcNac group can be further bound to second GlcNac group as part of the long-chain glycan structure. The second GlcNac group can, in one embodiment, be bound to other carbohydrate groups of the glycan structure including mannose or GlcNac.

In another embodiment, the β(1,2) xylose group is covalently linked to a mannose group which group can be bound to one or more other GlcNac groups as described above, for instance. The mannose group can be further covalently linked to other mannose groups (usually two(2)) in a branched structure, which groups can be further bound to GlcNac. The plant-derived rhTF could also be produced by plants that have been genetically modified to alter the nature of the post-translational glycosylation.

An exemplary long-chain glycan has the following structure:

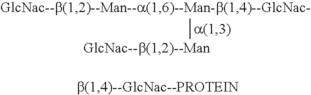

in which GlcNac is N-acetylglucosamine; and Man is mannose. See Ma., J K et al. Nature Reviews Vol. 4: 794 (2003) for additional information regarding illustrative long-chain glycan structures made by plants.

Also provided by the invention is a substantially purified preparation of the recombinant mammalian tissue factor disclosed herein. By the phrase “substantially pure” is meant molecules, either nucleic or amino acid sequences, that are removed from their natural environment, isolated or separated, and are at least 60% free, preferably 75% free and more preferably 90% free from other components with which they are naturally associated. Methods for making substantially pure proteins are disclosed herein and generally include centrifugation, chromatography, and related standard steps. An “isolated protein” is, therefore, a substantially purified protein.

Although not needed to practice the invention, the plant-derived tissue factor proteins disclosed herein can be “lipidated”, for instance with at least one of, preferably both, phosphatidylcholine and phosphatidylserine. Methods for lipidating tissue factor are known in the field and can be adapted as needed to lipidate the present proteins.

In the invention of this application, the TF protein is produced by plants and is associated with or bound to plant membranes. It is well known that “the biochemistry and cellular organization of fatty acid and glycerolipid synthesis in plant cells is quite different from the animal paradigm” (Miquel and Browse (1992) J Biochem 267:1502-1509; and references cited therein). A comparison of the composition of plant cell versus animal cell membranes indicates that there are some similarities, but also numerous differences (see reviews on plant membranes—J. B. Mudd (1980) “Phospholipid Biosynthesis” pp. 249-282 in The Biochemistry of Plants in P. K. Stumpf, ed. Academic Press, New York; mammalian membranes—H. Hauser & G. Poupart (1992) “Lipid Structure” pp. 3-71 in The Structure of Biological Membranes P. Yeagle, ed. CRC Press, Boca Raton, Fla.). Specifically, while both mammalian and plant membranes contain substantial amounts of phosphatidylcholine (PC) and phosphatidylethanolamine (PE), mammalian membranes contain higher proportions (on the order of 7-17%) of phosphatidylserine (PS), whereas plant membranes contain very little (1% or less). In addition, the fatty acids in plant membranes rarely have chain lengths greater than 18 carbon units, whereas the fatty acids in mammalian membranes can have longer chain lengths. The fatty acids in plant membranes tend to be more unsaturated with 18:3 (18 carbon chain length: 3 double bonds) predominating in plants, but seen only in trace amounts in mammalian membranes.

Given the differences in the membranes and glycosylation with mammalian cells compared to plants, it was interesting to find that, in the invention of this application, the TF preparations in which the TF protein is in association with plant membranes were at least as active as TF protein purified from plants expressing TF and relipidated using PC (75%) and PS (25%) (van t'Veer, et al. (1997) J. Biol. Chem. 272:7983-7994). This was demonstrated using the TF preparations to stimulate clotting in the Prothrombin Time in vitro assay (shown in Example 5).

As mentioned, the invention provides for transgenic plants that express tissue factor or a functional fragment thereof, preferably human tissue factor or a functional fragment thereof. One or a combination of standard methods for expressing heterologous proteins in plants can be used to express the tissue factor protein or fragment as needed.

For instance, a large number of particular plant production systems have been developed. These include expressing protein on oil bodies (Rooijen et al., (1995) Plant Physiology 109:1353-61; Liu et al., (1997) Molecular Breeding 3:463-70), through rhizosecretion (Borisjuk et al., (1999) Nature Biotechnology 17:466-69), in seed (Hood et al., (1997) Molecular Breeding 3:291-306; Hood et al., (1999) In Chemicals via Higher Plant Bioengineering (ed. Shahidi et al.) Plenum Publishing Corp. pp. 127-148; Kusnadi et al., (1997) Biotechnology and Bioengineering 56:473-84; Kusnadi et al., (1998) Biotechnology and Bioengineering 60:44-52; Kusnadi et al., (1998) Biotechnology Progress 14:149-55; Witcher et al., (1998) Molecular Breeding 4:301-12), as epitopes on the surface of a virus (Verch et al., (1998) J. Immunological Methods 220:69-75; Brennan et al., (1999) J. Virology 73:930-38; Brennan et al., (1999) Microbiology 145:211-20), and by stable expression of proteins in potato tubers (Arakawa et al., (1997) Transgenic Research 6:403-13; Arakawa et al., (1998) Nature Biotechnology 16:292-97; Tacket et al., (1998) Nature Medicine 4:607-09). Recombinant proteins can also be targeted to the seeds, chloroplast or secreted to identify the location that gives the highest level of protein accumulation. Each of these could be adapted to express the tissue factor or fragment in a suitable plant host.

Additional general methods for expressing proteins in plants have been reported. See PCT/US02/23624 to Bascomb, N. et al.; and PCT/US02/17927 to Hall, G. et al. These could be readily adapted to express the tissue factor protein or fragment in, for instance, Arabadopsis as well as a variety of other plants.

Further methods for expressing heterologous proteins in monocotyledenous and dicotyledenous plants have been reported. These include approaches that result in stable and constitutive expression of the protein of interest: 1) Agrobacterium-mediated gene transfer; 2) direct DNA uptake including methods for direct uptake DNA into protoplasts; 3) DNA uptake induced by brief electric shock of plant cells, 4) DNA injection into plant cells or tissues by particle bombardment, by the use of micropipette systems or by the direct incubation of DNA with germinating pollen; and 5) the use of plant virus as gene vectors. One or a combination of these methods can be used to create plants that express tissue factor and functional fragments thereof.

See e.g., Paszkowski, J., et al. (1989) in Cell Culture and Somatic Cell Genetics of Plants, Vol. 6, Molecular Biology of Plant Nuclear Genes eds. Schell, J., and Vasil, L. K., Academic Publishers, San Diego, Calif. p. 52-68; Klein, T. M., et al. in Progress in Plant Cellular and Molecular Biology, eds. Nijkamp, H. J. J., Van der Plas, J. H. W., and Van Aartrijk, J., (1988) Kluwer Academic Publishers, Dordrecht, p. 56-66; DeWet, J. M. J., et al. (1985) In Experimental Manipulation of Ovule Tissue, eds. Chapman, G. P. and Mantell, S. H. and Daniels, W. Longman, London, p. 197-209; Zhang, H. M. et al., (1988) Plant Cell Rep. 7:379-384; Frornn, M. E. et al., (1986) Nature 319:791-793; Hess, D. (1987) Int. Rev. Cyto. 107:367-395; Klein, T. M. et al., (1988) Bio/Technology 6:559-563; Neuhaus G. et al., (1987) Theor. Appl. Genet. 75:30-36; Neuhaus, G. and Spangenberg, G., (1990) Physiol. Plant. 79:213-217; Ohta, Y. (1986) Proc. Natl. Acad. Sci. USA 83:715-719; and US Published Patent Application 2003/0138456.

Agrobacterium-Mediated Gene Transfer

Gene transfer by means of engineered Agrobacterium strains has become routine for most dicotyledonous plants and for some monocotyledonous plants. See e.g., Fraley et al. (1983) Proc. Natl. Acad. Sci. USA 80:4803; Watson et al. (1985) EMBO J. 4:277; Horsch et al. (1985) Science 227:1229; Hernalsteens et al. (1984) EMBO J. 3:3039; Comai et al. (1984) Nature (London) 317:741; Shah et al. (1986) Science 233:478; Bytebier et al. (1987) Pro. Natl. Acad. Sci. USA 84:5345; Schafew et al. (1987) Nature 327:529; Potrykus, I. (1990) Biotechnol 8:535; Grimsley et al. (1987) Nature 325:177; Gould et al (1991) Plant Physiol 95:426; Ishida et al. (1996) Nature Biotechnology 14:745; and U.S. Pat. No. 5,591,616, and the references cited therein.

An appropriate Agrobacterium strain utilized in the methods of the invention is modified to contain a gene or genes of interest, or a nucleic acid to be expressed in the transformed cells. The nucleic acid to be transferred is incorporated into the T-region and is flanked by at least one T-DNA border sequence. A variety of Agrobacterium species are known particularly for dicotyledon transformation. Such Agrobacterium can be used in the methods of the invention. See, for example, Hooykaas, P. J. (1989) Plant Mol. Biol. 13:327; Smith et al. (1995) Crop Science 35:301; Chilton, M. O. (1993) Proc. Natl. Acad. Sci. USA 90:3119; Mollony et al. N:Monograph Theor Appl Genet NY, Springer verlag 19:148, 1993; and Ishida et al. (1996) Nature Biotechnol. 14:745; Komari, T. et al. (1996) The Plant Journal 10:165.

In the Ti plasmid, the T-region is distinct from the vir region whose functions are responsible for transfer and integration. Binary vector systems have been developed where the manipulated disarmed T-DNA carrying foreign DNA and the vir functions are present on separate plasmids. In this manner, a modified T-DNA region comprising foreign DNA (the nucleic acid to be transferred) is constructed in a small plasmid which replicates in E. coli. This plasmid is transferred conjugatively in a tri-parental mating or via transformation into A. tumefaciens which contains a compatible plasmid-carrying virulence gene. The vir functions are supplied in trans to transfer the T-DNA into the plant genome. Such binary vectors are useful in the practice of the present invention. Disclosure relating to making and using a variety of Ti vectors has been published. See U.S. Pat. Nos. 5,276,268; 5,283,184; and 5,286,635 for examples of suitable Ti vectors for use with the invention.

Further vectors for use with the invention include those “super-binary” disclosed in U.S. Pat. No. 5,591,616 and EPA 0604662A1, for instance. See also Hood et al. (1984) Biotechnol. 2:702-709; Hood et al. (1986) J. Bacteriol. 168:1283-1290; Komari et al. (1986) J. Bacteriol. 166:88-94; Jin et al. (1987) J. Bacteriol. 169:4417-4425; Komari T. (1989) Plant Science 60:223-229; ATCC Accession No. 37394).

Other appropriate vectors that can be used such as those described by deFramond, A. et al., (1983) Bio/Technology 1:263; An, G. et al., (1985) EMBO J. 4:277; and Rothstein, S. J. et al., (1987) Gene 53:153.

Other sequences for facilitating site-specific genome integration and/or controlled excision and/or reinsertion into the genome may also be provided. For example, the Cre/lox system can be used to obtain targeted integration of an Agrobacterium T-DNA at a lox site in the genome of Arabidopsis. Site-specific recombinants, and not random events, are preferentially selected by activation of a silent lox-neomycin phosphotransferase (nptII) target gene. Cre recombinase can be provided transiently by using a co-transformation approach. See, e.g., as described in Vergunst, et al., (1998) Plant Mol Biol 38(3):393-406.

A vector suitable for chloroplast transformation can be used in accord with the invention as well. Chloroplasts are prokaryotic compartments inside eukaryotic cells. Since the transcriptional and translational machinery of the chloroplast is similar to E. coli (Brixey et al., (1997) Biotechnology Letters 19:395-400), it is possible to express prokaryotic genes at very high levels in plant chloroplasts than in the nucleus (Daniell, et al. (2002) Trends in Plant Science 7:84-91; Tregoning, et al. Nucleic Acid Res. 31:1174-1179). In addition, plant cells contain up to 50,000 copies of the circular plastid genome (Bendich (1987) Bioessays 6:279-282) which may amplify a recombinant gene like a plasmid, enhancing levels of expression. In some invention embodiments, chloroplast expression may be a hundred-fold higher than nuclear expression in transgenic plants (Daniell, WO 99/10513).

Additionally suitable vectors for use with the invention are replicable, preferably in a microbial host such as a bacterium (e.g., E. coli) and include the following components: (a) a first promoter operable in a plant; (b) an expressible nucleic acid sequence encoding a mammalian tissue factor protein or functional fragment thereof, wherein the nucleic acid sequence is operatively associated with the promoter; and (c) a termination sequence operatively associated with the nucleic acid sequence.

The term “promoter” refers to the nucleotide sequences at the 5′ end of a structural gene which directs the initiation of transcription. Generally, promoter sequences are necessary, but not always sufficient, to drive the expression of a downstream gene. In the construction of heterologous promoter/structural gene combinations, the structural gene is placed under the regulatory control of a promoter such that the expression of the gene is controlled by promoter sequences. The promoter is positioned preferentially upstream to the structural gene and at a distance from the transcription start site that approximates the distance between the promoter and the gene it controls in its natural setting. As is known in the art, some variation in this distance can be tolerated without loss of promoter function. As used herein, the term “operatively linked” means that a promoter is connected to a coding region in such a way that the transcription of that coding region is controlled and regulated by that promoter. Means for operatively linking a promoter to a coding region are well known in the art.

Illustrative Plant Promoters

The gene constructs used may include all of the genetic material and such things as promoters, IRES elements, etc. These expression cassettes can either require some external stimuli to induce expression, such as the addition of a particular nutrient or agent, change in temperature, etc. or can be designed to express an encoded protein immediately and/or spontaneously during growth.

Thus, the expression of a gene encoding a desired protein may be controlled by constitutive or regulated promoters. Regulated promoters may be tissue-specific, developmentally regulated or otherwise inducible or repressible, provided that they are functional in the plant cell. Regulation may be based on temporal, spatial or developmental cues, environmentally signaled, or controllable by means of chemical inducers or repressors and such agents may be of natural or synthetic origin and the promoters may be of natural origin or engineered. Promoters also can be chimeric, i.e., derived using sequence elements from two or more different natural or synthetic promoters.

Preferably, a promoter used in the construct yields a high expression level of the gene, allowing for accumulation of the protein to be at least about 0.1-1%, at least about 1-5%, and more preferably, at least about 5% of total soluble protein, and/or yields at least about 0.1%, preferably at least about 0.5%, and most preferably, at least about 1%, of the total intercellular fluid (ICF) extractable protein.

The promoter should preferentially allow expression in all of the plant tissues, but most preferably, in all of the leaf, stem and root tissue. Additionally, or alternatively, the promoter allows expression in floral and/or seed tissue. In the present invention, the Arabidopsis Actin 2 promoter, the (OCS)3MAS promoter and various forms thereof, the CaMV 35S, and figwort mosaic virus 34S promoter are preferred. However, other constitutive promoters can be used. For example, the ubiquitin promoter has been cloned from several species for use in transgenic plants (e.g., sunflower (Binet et al., (1991) Plant Science 79:87-94; and maize (Christensen et al., (1989) Plant Molec. Biol. 12:619-632). Further useful promoters are the U2 and U5 snRNA promoters from maize (Brown et al., (1989) Nucleic Acids Res. 17:8991) and the promoter from alcohol dehydrogenase (Dennis et al., (1984) Nucleic Acids Res. 12:3983).

In another aspect, a regulated promoter is operably linked to the gene. Regulated promoters include, but are not limited to, promoters regulated by external influences (such as by application of an external agent, e.g., such as chemical, light, temperature, and the like), or promoters regulated by internal cues, such as regulated developmental changes in the plant. Regulated promoters are useful to induce high-level expression of a desired gene specifically at, or near, the time of harvest. This may be particularly useful in cases where the desired protein limits or otherwise constrains growth of the plant, or is in some manner, unstable.

Plant promoters which control the expression of transgenes in different plant tissues by methods are known to those skilled in the art (Gasser & Fraley, (1989) Science 244:1293-99). The cauliflower mosaic virus 35S promoter (CaMV) and enhanced derivatives of CaMv promoter (Odell et al., (1985) Nature, 3(13):810), actin promoter (McElroy et al., (1990) Plant Cell 2:163-71), AdhI promoter (Fromm et al., (1990) Bio/Technology 8:833-39, Kyozuka et al., (1991) Mol. Gen. Genet. 228:40-48), ubiquitin promoters, the Figwort mosaic virus promoter, mannopine synthase promoter, nopaline synthase promoter and octopine synthase promoter and derivatives thereof are considered constitutive promoters. Regulated promoters are described as light inducible (e.g., small subunit of ribulose biphosphatecarboxylase promoters), heat shock promoters, nitrate and other chemically inducible promoters (see, for example, U.S. Pat. Nos. 5,364,780; 5,364,780; and 5,777,200).

Tissue specific promoters are used when there is reason to express a protein in a particular part of the plant. Leaf specific promoters may include the C4PPDK promoter preceded by the 35S enhancer (Sheen, (1993) EMBO, 12:3497-505) or any other promoter that is specific for expression in the leaf. For expressing proteins in seed, the napin gene promoter (U.S. Pat. Nos. 5,420,034 and 5,608,152), the acetyl-CoA carboxylase promoter (U.S. Pat. Nos. 5,420,034 and 5,608,152), 2S albumin promoter, seed storage protein promoter, phaseolin promoter (Slightom et. al., (1983) Proc. Natl. Acad. Sci. USA 80:1897-1901), oleosin promoter (Plant et al., (1994) Plant Mol. Bio. 25:193-205; Rowley et. al., 1997, Biochim. Biophys. Acts 1345:1-4; U.S. Pat. No. 5,650,554; PCT WO 93/20216), zein promoter, glutelin promoter, starch synthase promoter, and starch branching enzyme promoter are all useful.

Plant promoters can be selected to be inducible or constitutive as needed to achieve an intended invention result.

Generally, any plant expressible genetic construct is suitable for use in the methods of the invention. Particular promoters may be selected in consideration of the type of recombinant protein being expressed.

Other regulatory elements such as enhancer sequences also may be provided. For example, in one aspect, expression cassettes that contain multimerized transcriptional enhancers from the cauliflower mosaic virus (CaMV) 35S gene are used. See, e.g., Weigel, et al. (2000) Plant Physiol 122(4):1003-13.

If desired, suitable vectors for use with the invention can further include a sequence that suppresses gene silencing in plants. Such sequences are generally derived from plant viruses and have been disclosed. See Anandalakshimi, R. et al. (1998) PNAS (USA) 95: 13079.

Illustrative Termination Sequences

In the vector described in this invention the nopaline synthase gene (Nos) terminator is used. Other terminators include e.g., octopine synthase gene (OCS) terminator (Halfter, et al. (1992) Mol Gen Genet. 231:186-193) or the cruciferin A (cruA) terminator (Rodin, et al. (1992) Plant Mol Bio 20:559-563) can be used.

Selectable Markers and/or Reporter Genes

Selectable markers, such as antibiotic (e.g., kanamycin (nptII) and hygromycin (hpt)) resistance, herbicide (glufosinate, imidazlinone, glyphosate, AHAS, EPSPS) resistance or physiological markers (visible or biochemical) are used to select cells transformed with the nucleic acid construct. Non-transgenic cells (i.e., non-trans formants) on the other hand, are either killed or preferentially do not grow under the selective conditions. In one aspect, a selectable marker gene is a gene which encodes a protein providing resistance or physiological markers. However, in another aspect, a selectable marker gene is a gene encoding an antisense nucleic acid.

Reporter genes may be included in the construct or they may be contained in the vector that ultimately transports the construct into the plant cell. As used herein, a “reporter gene” is any gene which can provide a cell in which it is expressed with an observable or measurable phenotype.

Expression of reporter genes yields a detectable result, e.g., a visual colorimetric, fluorescent, luminescent or biochemically assayable product; a selectable marker, allowing for selection of transformants based on physiology and growth differential; or display a visual physiologic or biochemical trait. Commonly used reporter genes include lacZ (β-galactosidase), GUS (β-glucuronidase), GFP (green fluorescent protein and mutated or modified forms thereof), luciferase, or CAT (chloramphenicol acetyltransferase), which are easily visualized or assayable. Such genes may be used in combination or instead of selectable markers to enable one to easily pick out clones of interest. In one aspect, a selectable marker gene is a gene encoding a protein product.

Selectable markers can also include molecules that facilitate isolation of cells which express the markers. For example, a selectable marker can encode an antigen which can be recognized by an antibody and used to isolate a transformed cell by affinity-based purification techniques or by flow cytometry. Reporter genes also may comprise sequences which are detected by virtue of being foreign to a plant cell (e.g., detectable by PCR, for example). In this embodiment, the reporter gene need not express a protein or cause a visible change in phenotype.

In a more particular embodiment of the previously mentioned replicable vector, the vector further includes d) a second promoter operable in the plant, (e) a selectable marker gene operatively associated with the second promoter, (f) an Agrobacterium TR sequence, and (g) an Agrobacterium TL sequence in which the expressible nucleic acid sequence encoding the mammalian tissue factor protein or fragment further encodes a plant specific signal peptide operatively linked to the tissue factor protein or fragment. The expressible nucleic acid sequence encoding the mammalian tissue factor or fragment is typically at least about 70% identical to the mature human TF sequence shown in Table I as SEQ ID NO. ______, preferably at least about 80%, about 90%, or at least about 99% or 100% identical to that sequence. Alternatively, or in addition, the encoded mammalian tissue factor or fragment exhibits at least about 70% of the prothrombin time of the mature human TF sequence shown in Table I as SEQ ID NO. ______ as monitored in a standard prothrombin time (PT) assay.

By the phrase “standard prothrombin time” assay is meant the assay generally described by Miletich, J P (1995) in Prothrombin Time (Chapter L33) in Williams Hematology, 5^{th} Ed. (Beutler, E. et al. Eds.) McGraw-Hill, Inc., Health Professions Div., New York, pages L82-L84.

Generally, the standard prothrombin time assay includes the steps of diluting a tissue factor sample (e.g., crude plant extract that includes TF) in an acceptable buffer to make a serial dilution (about 0.2 mL total). A preferred buffer is PT buffer (see Example 5). The assay can be initiated by adding about 0.1 mL of blood plasma, preferably pre-incubated with the buffer. Coagulation can be monitored by one or a combination of different strategies such as use of an automated coagulation timer. Examples of such devices are generally available from commercial vendors.

A specific example of the standard prothrombin time assay is provided in Example 5.

As discussed, the invention is flexible and can be used to prepare one or a variety of transgenic plants (dicots and certain monocots) that express mammalian tissue factor (or functional fragment). An illustrative plant is a member of the family Fabaceae, Solanaceae, Brassicaceae, Rosaceae or Compositae. More specific plants include the following: duckweed, tobacco (Nicotiana tabacum), mustard (Arabidopsis), potato (Solanum tuberosum), soybean (glycine max), tomato (Lycopersicon esculentum), cassaya (Manihot esculenta), beets, peanuts (Arachis hypogaea), cotton (Gossypium hirsutum), citrus trees (Citrus spp.), corn or maize (Zea mays), beans (e.g., green beans (Phaseolus vulgaris) and lima beans (Phaseolus limensis)), peas (Lathyrus spp.), sugarbeet, sunflower, carrot, celery, flax, cabbage (and other cruciferious plants), pepper, mango, peach, apple, pear, banana, sunflower, onion, strawberry, alfalfa, oat, wheat, rye, rice, barley, sorghum and canola.

Additional plants include those of the genus Lactucua and particularly L. sativa (lettuce). Alternatively, a suitable cereal plant can be used in accord with the invention.

Also envisioned are transgenic plant progeny obtained from the cutting, slip, cultivar, or seed of the foregoing transgenic plants in which the progeny plant also includes the expressible nucleic acid sequence encoding the mammalian tissue factor protein or functional fragment thereof.

As discussed in the Example section, it will often be use L. sativa (lettuce) as the plant of choice for expressing the mammalian tissue factor or fragment. In this invention embodiment, the protein can be expressed stably or transiently as needed to achieve an intended result. Lettuce will be a plant of choice particularly in embodiments in which large-scale production of the protein is desirable. The fleshy, large leave and relatively simple growth requirements, and other characteristics will serve this objective. As discussed below in more detail, it has also been found that lettuce is an especially amenable for the transient expression of tissue factor. If desired however, lettuce can be used to stably express the tissue factor or a functional fragment using, for instance, suitable transformation methods described herein.

Accordingly, the invention provides in one embodiment, a transgenic lettuce (L. sativa) that includes transformed plant cells in which the cells comprise at least one and preferably all of the following:
(a) a first promoter operable in a plant,
(b) an expressible nucleic acid sequence encoding a mammalian tissue factor protein or functional fragment thereof, wherein the nucleic acid sequence is operatively associated with the promoter; and
(c) a termination sequence operatively associated with the nucleic acid sequence.
(d) a second promoter operable in the plant,
(e) a selectable marker gene operatively associated with the second promoter,
(f) an Agrobacterium TR sequence, and
(g) an Agrobacterium TL sequence.

Preferably, the expressible nucleic acid sequence encoding the mammalian tissue factor protein or fragment further encodes a plant specific signal peptide operatively linked to the tissue factor protein or fragment. In a more particular embodiment of the transgenic lettuce: i) the first promoter is inducible or constitutive; ii) the expressible nucleic acid sequence encodes the mature human tissue factor sequence shown in Table I as SEQ ID NO. ______, a functional fragment thereof or a sequence at least about 70% identical to the mature human tissue factor sequence shown in Table I as SEQ ID NO. ______ (preferably at least about 80% or 90%) and exhibiting at least about 70% of the prothrombin time (PT) of the tissue factor protein shown in Table I as the mature human tissue factor protein (preferably at least about 80% or 90%) as determined by the standard PT assay; iii) the termination sequence is Nos-3′; iv) the second promoter is a 35S promoter; and v) the selectable marker gene is NPTII.

More preferred expressible nucleic acid sequences for use with this invention embodiment encode human tissue factor sequence with about the same activity in the PT assay as the mature human tissue factor sequence exemplified in Table I as SEQ ID NO. ______.

Transformation of Lettuce

In embodiments in which use of lettuce is preferred, nearly any of the transformation methods provided herein can be used to produce transgenic plants that express the tissue factor. Such expression can be stable or transient as needed. One or a combination of the methods described in PCT/US02/17927 can be used including those relating to vacuum-infiltration and incubation of Agrobacterium-treated plant tissue. A specific protocol for transforming lettuce heads transiently is provided in the Examples.

Crude Plant Extracts that Include TF

As discussed, it is an object of the present invention to provide crude extracts of the transgenic plants disclosed herein which extracts will include mammalian tissue factor, preferably human tissue factor; or a functional fragment thereof. As also discussed, it has been surprisingly found that plants can express tissue factor that is biologically active. Thus it is a specific objective to provide such active tissue factor in crude form, e.g., in medical, veterinary or combat settings where functioning tissue factor is needed quickly and/or in large amounts regardless of purity.

By the phrase “crude extract” is meant an extract or lysate of plant tissue (leaves, stalks, flowers, roots, tubers, bulbs, fruits, seeds, etc) that is prepared by contacting the plant tissue with a pharmaceutically acceptable solution (water, phosphate-buffered saline (PBS), etc.), substantially disrupting the tissue (e.g., by use of a morter and pestle, french press, blender and the like) in the presence of between from about 1 to about 10 volumes of solution, and then removing debris by filtration or gentle centrifugation, typically about 1,000×g for less then about 30 minutes, preferably between from about 5 to about 20 minutes. Of course, the precise amount of solution will add will depend on recognized parameters such as the plant selected and the concentration of tissue factor desired in the extract. The debris is removed and the remaining solution kept as the “crude extract”. In most embodiments, one or a combination of pharmaceutically acceptable buffers are preferred (such as PBS) to help preserve tissue factor integrity and maximize potential therapeutic use. A variety of pharmaceutically acceptable solutions have been disclosed. See Remington's Pharmaceutical Sciences, (Mack Publishing Co., Easton Pa., (1980)).

As mentioned, the crude extract of the invention can provide mammalian tissue factor or a functional fragment thereof at relatively low cost and without time consuming relipidation. Crude extract is relatively stable and may be stored at low temperature (below freezing, e.g., −70 C) until needed.

Typical crude extracts in accord with the invention will have between about 1 ng/mL to 1 μg/mL of the mammalian tissue factor protein or functional fragment, or preferably between from about 1 μg/mL to 100 μg/mL.

Tissue Factor Isolation From Crude Extract

Although the mammalian tissue factor in the crude extract is biologically active i.e., it shows good activity in the standard PT assay without any relipidation, it will sometimes be useful to make a more purified tissue factor preparation. In some instances, a substantially pure preparation will be helpful.

In these embodiments and after cultivation, the transgenic plant is harvested to recover the produced multi-subunit protein or processed protein (and/or other proteins produced by structural genes according to the invention). This harvesting step may comprise harvesting the entire plant, or only the leaves, or roots or cells of the plant. This step may either kill the plant or, if only the portion of the transgenic plant is harvested, may allow the remainder of the plant to continue to grow.

After harvesting, protein isolation may be performed using methods routine in the art. For example, at least a portion of the plant may be homogenized, and the protein extracted and further purified. Extraction may comprise soaking or immersing the homogenate in a suitable solvent. As discussed above, proteins may also be isolated from interstitial fluids of plants, for example, by vacuum infiltration methods, as described in U.S. Pat. No. 6,284,875. Purification methods include, but are not limited to, immuno-affinity purification and purification procedures based on the specific size of a protein/protein complex, electrophoretic mobility, biological activity, and/or net charge of the multimeric protein to be isolated. Acceptable purification methods thus include at least one of chromatography, centrifugation, and immunoprecipitation (see U.S. Pat. Nos. 5,986,065 and 6,555,319 for illustrative anti-tissue factor antibodies).

In a more particular method, the crude extract is centrifuged at about 20,000×g followed by gentle filtering through a 1 micron filter to yield a supernatant. That supernatant is subjected to chromatography (preferably Q Sepharose Fast Flow Resin). The flow through was subjected to affinity chromatography (preferably 147 affinity) followed by washing with an acceptable wash buffer. Flow through can be stored at low temperature directly or subjected to standard buffer exchange procedures. See the Examples below.

As mentioned, it is another object of the invention to provide coagulant compostions that include plant-derived mammalian tissue factor and fragments disclosed herein. Such compositions can include, for instance, the crude extract. Alternatively, and when more purified TF is needed, the coagulants and coagulant compositions can include a substantially pure preparation of the tissue factor or functional fragment thereof.

Coagulant Formulations

The coagulant compositions of the invention can be employed, either alone or in combination with one or more other therapeutic agents or procedures that help minimize blood flow. Such agents include other blood factors (e.g., tissue factor fragments) or even solid supports such as cotton gauze. Illustrative techniques include applying pressure to the wound, cauterization or the use of sutures. In this regard, a preferred coagulant composition will include one or more conventional excipients, i.e., pharmaceutically acceptable organic or inorganic carrier substances suitable for parenteral, enteral or intranasal application which do not deleteriously react with the active compounds and are not deleterious to the recipient thereof.

Suitable pharmaceutically acceptable carriers include but are not limited to water, salt solutions, alcohol, vegetable oils, polyethylene glycols, gelatin, lactose, amylose, magnesium stearate, talc, silicic acid, viscous paraffin, perfume oil, fatty acid monoglycerides and diglycerides, petroethral fatty acid esters, hydroxymethyl-cellulose, polyvinylpyrrolidone, etc. The pharmaceutical preparations can be sterilized and if desired mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously react with the active compounds (typically the tissue factor). See Remington's Pharmaceutical Sciences, infra.

Typical amounts of crude extract and/or more purified tissue factor to use in a particular coagulant compositions will vary according to understood parameters such as intended use of the composition, the amount of blood flow anticipated, the particular tissue factor sequence selected, the rapidity of blood flow cessation needed etc. However in most instances, the amount of tissue factor or functional fragment in the compositions (expressed as w/w) will be between from about 1 nanogram to about 5000 micrograms per milliliter of blood, for instance about 100 nanograms to about 50 micrograms.

Treatment Methods and Other Uses

The coagulant compositions produced in accord with the invention can be used in a wide variety of setting including diagnostic assays (PT, aPTT and other standard blood clotting tests). In this embodiment, the coagulant composition can be used in place of relipidated TF.

However, it will often be preferred to use the coagulant composition to prevent or treat blood loss in a subject. In this instance, the method will include contacting the subject with a therapeutically effective amount of the anticoagulants disclosed herein. Alternatively, the method can be used with any of the crude extracts provided herein as well as the substantially pure mammalian tissue factor or functional fragments.

In addition to its role in blood coagulation, TF plays a role in angiogenesis. This was discovered when it was found that mice in which TF had been genetically knocked out were unable to develop beyond embryonic day 9-10 due to the failure to develop blood vessels (Carmeliet, et al (1996) Nature 383:73-75; Bugge, et al. (1996) PNAS 93:6258-6263; Toomey, et al. (1996) Blood 88:1583-1587). Further studies have demonstrated that activation of coagulation proteases can lead to activation of the protease activated receptors leading to increased production of vascular endothelial growth factors which stimulate angiogenesis (Richard, et al. (2002) Oncogene 20:1556-1562; Milia, et al. (2002) Circ. Res. 91:346-352). In addition, the over-expression of TF on tumor cells promotes tumor growth, vascularization and metastasis (Mueller, et al. (1992) PNAS 89:11832-11836).

The roles of TF in promoting blood coagulation, angiogenesis and vascular development/remodeling are well documented. These roles are clearly important aspects of wound repair and healing. Several reports have indicated that TF promotes wound repair and healing using TF isolated from mammalian sources or produced by recombinant means in hosts other than plants (Nakagawa, et al. (1998) Seminars in Thromb. & Hemostasis 24:207-210; Philippart, et al. (2003) The Internatl J of Oral & Maxillofacial Implants 3:411-416). To further study the effects on coagulation and wound healing in vivo, an animal model was used in which partial thickness wounds were made on the back of a pig (Eaglstein and Mertz (1978) J Invest. Dermatology 71:382-384; Alvarez, Mertz & Eaglstein (1983) J. Surgical Res. 35:142-148). Using this model, it was shown that the plant-derived TF effectively stopped bleeding from the wounds faster than controls and at least comparably to purified TF protein that had been relipidated (shown in an Example).

In addition, the invention provides a method for promoting wound healing in a subject. In one embodiment, the method includes contacting the subject with a therapeutically effective amount of any of the coagulant compositions disclosed herein.

Further methods in accord with the invention include promoting angiogenesis in a subject. In one embodiment, the method includes contacting the subject with a therapeutically effective amount of any of the anticoagulant compositions disclosed herein.

Further invention methods include promoting vascular remodeling in a subject. In one embodiment, the method includes contacting the subject with a therapeutically effective amount of any of the anticoagulant compositions disclosed herein.

The coagulant compositions can be used in the methods in nearly any acceptable form such as pouring or spaying the coagulant composition on or near a wound.

The invention also provides an absorbent (or adsorbent) that includes at least one of the coagulant compostions disclosed herein. Preferably, the absorbent is a medically acceptable support such as gauze (preferably sterile and including cotton or a cotton blend). Nearly any of the coagulant compositions can be administered to the absorbent e.g., as a dry formulation or in wet or moist form.

Also provided is an adhesive support that includes the absorbent. Preferably, the adhesive support is sterile. If desired, the adhesive support and absorbent can be placed or sealed into a wrapper container that is preferably impervious to water and air.

The following describes particular invention embodiments. It should not be construed as limiting the scope of the invention.

In one embodiment, this invention relates to the production of active recombinant human tissue factor (rhTF) in plants. It features an efficient and inexpensive way of production of biomass possessing rhTF, which can be used directly for bleeding cessation and wound healing. No purification or lipidation are required. A binary vector was constructed with the gene coding for full-length (263 aa) rhTF, which can be used both for transient expression and for stable transformation in plants, plant tissues or plant cells. In one example, the rhTF was produced transiently in lettuce. Two samples of this protein were prepared and tested. One sample of plant-derived rhTF was purified and lipidated (plant purified TF). A second sample was a fraction of a crude extract that had not been purified or lipidated artificially. The fraction was made by simply homogenizing leaf biomass with an equal volume of PBS which was then centrifuged for 5 min at 1,000×g and 4° C. Supernatant was used as the crude extract. Both samples were tested for blood coagulation activity using prothrombin time assay. It was found that both samples possessed a high coagulation activity close to human TF. Animal study on pig model also demonstrated notable coagulation activity. Thus, using plant expression system one can produce inexpensive and highly active rhTF, which could be used both for bleeding cessation and wound healing with humans and other mammals.

In one embodiment, one can use cells of Agrobacterium tumefaciens bearing a binary vector with the gene for rhTF. This vector can be used both for transient expression and for stable transformation of plants, plant tissues or plant cells. In this vector, the eukaryotic segment comprises: TL (the left terminal repeat of the tumor-inducing region from the Agrobacterium TI plasmid), a promoter (inducible or constitutive), apoplast targeted signal peptide, gene of rhTF, termination transcription signal, a 35S promoter directing expression of NPTII gene, termination transcription signal, and TR (the right terminal repeat of the tumor-inducing region from the Agrobacterium TI plasmid).

In another embodiment, rhTF gene will be expressed transiently or in stable transgenic plants.

In another embodiment, preparations comprising the rhTF protein can be obtained which are active without any purification procedure.

In another embodiment, the rhTF fractions can be formulated in a liquid form, a lyophilized form for reconstitution or as a dry powder aerosolized application, as creams or other topical formulations, or as a powdered leaf biomass.

In another embodiment, such formulations can be used for bleeding cessation and wound healing in humans or other mammals.

As discussed, the invention features, in one embodiment, a novel technology of production and application of rhTF for prevention of intensive bleeding at wounded people or mammalian animals as well as for acceleration of the wound healing. It allows produce unlimited quantities of inexpensive source of rhTF which can be applied for wound treatments.

Further provided is a composition comprising soluble, functional human tissue factor produced by and obtained from transgenic plants is disclosed. Methods are also disclosed for the expression of genes for human tissue factor in transgenic plants, plant tissue or plant cells. More specifically, active tissue factor preparations comprising fractions of crude extracts from plant material containing the tissue factor protein can be made, as well as preparations in which the tissue factor is purified and relipidated. Furthermore, the fractions from crude extracts of plant material expressing tissue factor are active without requiring the introduction of exogenous lipids. Such preparations are active, as demonstrated by triggering coagulation in vitro and halting bleeding in vivo. Also disclosed are methods of use of such preparations for promoting blood coagulation, minimizing blood loss and promoting wound closure and healing.

The following Examples are intended to be illustrative and not limiting of the present invention.

### EXAMPLE 1

### Preparation of Agrobacterium Vector Encoding Tissue Factor

This example describes the method of producing recombinant human tissue factor (rhTF) in plants. The method involves cloning the DNA sequence coding for tissue factor into a plasmid DNA vector that is capable of replication in Escherichia coli and Agrobacterium tumifaciens. This recombinant DNA molecule can be introduced into plants, plant tissues or cells, such that the TF gene is expressed.

Binary Vector Construction

The binary vector was constructed so as to have a gene encoding full-length (263 aa) rhTF (FIG. 1), which can be used both for transient expression and for stable transformation. In FIG. 2, the map of one binary vector is shown comprising: TR, Nos-3′ terminator (reverse orientation), gene of rhTF (reverse orientation) with tomato subtilase apoplast-targeting signal peptide (Janzik et al., 2000) (reverse orientation), 2,4-D inducible (OCS)3Mas promoter (Gelvin et al., U.S. Pat. No. 5,955,646) (reverse orientation), 35S promoter, NPTII gene, 35S-3′ terminator, and TL.

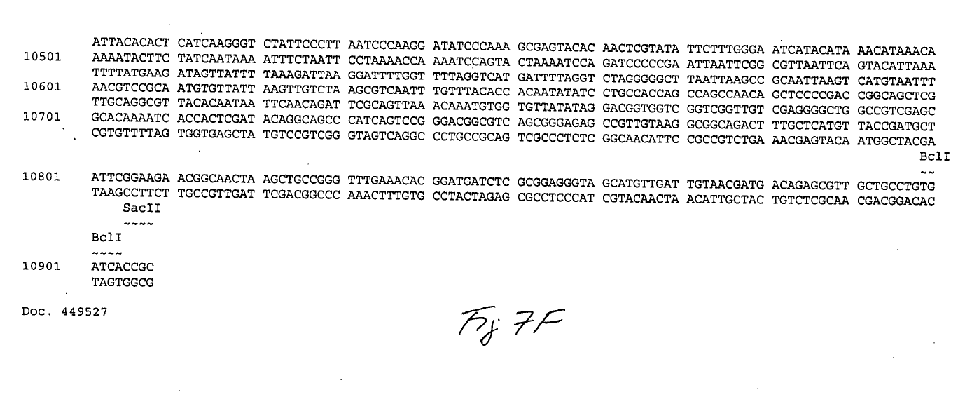

To construct the modified binary vector, the rhTF gene was copied from a plasmid containing the rhTF gene (or from a human cDNA) by polymerase chain reaction (PCR). Using PCR approach, the rhTF gene was fused to the tomato subtilase apoplast-targeting signal peptide (Janzik et al., 2000). A SalI site was introduced by PCR at the 5′-end of the PCR product and an SpeI site was introduced at the 3′-end. To transfer the modified rhTF sequence into plant binary vector, a vector pSUNP2, bearing a kappa light chain antibody (see U.S. Ser. No. 10/739,447) was cleaved with SalI and SpeI and ligated to the rhTF PCR product cleaved with the same endonucleases. Other binary plant transformation vectors could also have been used (for example, pBIN19 (Bevan, 1984), which was an precursor of pSUNP2, or pBIN20 (Hennegan and Danna, 1998). In replacing the SalI-SpeI fragment containing the kappa light chain with SalI-SpeI fragment containing the rhTF fused to the subtilase signal, the coding region was inserted between (OCS)3Mas promoter and Nos-3′ terminator. The resulting plasmid with rhTF was named pSUNP12 (a map is provided in FIG. 2) and a complete sequence of the vector is provided in FIGS. 7A-F. For transient expression of rhTF in lettuce the, the pSUNP12 vector was transferred into Agrobacterium tumefaciens strain C58 (Sciaky et al., 1978) or any related strains (for example, C58C1).

### EXAMPLE 2

### Preparation of Agrobacterium tumefaciens Suspension

Agrobacterium tumefaciens strain C58/C1 carrying the pSUNP12 vector were grown and vacuum-infiltrated into leaves of lettuce according to the procedure of transient expression in lettuce (U.S. Ser. No. 10/739,447).

The Agrobacterium culture with the pSUNP12 binary vector was grown on LB broth supplemented with 5 g/L sucrose, 2 mM MgSO₄, 10 mM MES pH 5.6 and 20 μM acetosyringone overnight at 29° C. The ratio of the inoculum used to start the overnight culture to final culture volume was 1:100 and gave a suspension with a final OD₆₀₀ of about 2.4. The suspension was supplemented with 10 mM MES, pH 5.6, and 200 μM acetosyringone and incubated for 1 hour at 22° C. Before infiltration, 100 μM of 2,4-D and 0.005% Tween 20 were added. Lettuce was vacuum-infiltrated with the Agrobacterium culture and the leaves were treated as described in U.S. Ser. No. 10/739,447 and Example 3. Lettuce leaves, transiently expressing rhTF, were used for isolation and purification of rhTF or alternatively, for preparation of crude extracts.

### EXAMPLE 3

### Transformation of Lettuce

Vacuum-Infiltration and Incubation of Treated Lettuce Heads

1.25 L of pretreated suspension of Agrobacterium was placed into 2 L glass beaker inside vacuum-dessicator. Whole heads of lettuce were immersed into suspension and held for 20 min under a vacuum (25″ column of water) and then, the vacuum was released rapidly. Lettuce heads were briefly rinsed in water and left for 3-4 days at 22° C. and 16 hours of daylight in boxes with transparent cover on wet paper towels. After incubation, the treated lettuce was homogenized for protein extraction then used for purification of the rhTF as described in Example 4.

### EXAMPLE 4

### Preparation of Plant rhTF Samples

Plant produced rhTF samples were prepared in purified form and as crude extract as follows:

1. Purification of lettuce produced rhTF: Leaf biomass was homogenized for 1 min at 4° C. in Waring blender at maximum speed, with 1:1 of extraction buffer (100 mM Tris-HCl, pH8.0; 1 mM EDTA; 0.15 M NaCl; 1% Triton X-100). Homogenate was centrifuged for 15 min at 7,000×g, then, supernatant was centrifuged for 15 min at 15,000×g. Final supernatant was frozen and stored at −70° C.

The frozen lettuce TF extraction was thawed at 4° C., and was centrifuged for 30 min at 20,000×g, followed by filtering the supernatant through 1 μm filter. Q Sepharose Fast Flow resin was equilibrated with 5 volumes of buffer (20 mM Tris-HCl, 0.1 M NaCl, 0.5% Tween 80, pH 7.5), then the supernatant was loaded onto the Q Sepharose resin and the unbound material was collected by vacuum. The Q flow-through was centrifuged at 20,000×g for 30 min, and the supernatant loaded onto an 147 affinity column pre-equilibrated with buffer (20 mM Tris-HCl, 0.1 M NaCl, 0.5% Tween 80, pH 7.5). After loading, the column was washed with 5 volumes of 147 column wash buffer 1 (20 mM Tris-HCl, 1 M NaCl, 0.5% glucoside, pH 8.0), buffer II (50 mM Na-phosphate, pH 6.0, 0.5% glucoside) and buffer III (100 mM Na-acetate, pH 4.0, 0.5% glucoside, 0.15 M NaCl), respectively, followed by eluting the column with 5 volumes of elution buffer (100 mM acetic acid, pH 3.0, 1% glucoside, 0.15 M NaCl). The protein peak was collected and adjusted to pH to 8.0 with 1 M Tris. The resulting TF was concentrate and buffer exchanged into storage buffer (20 mM Tris-HCl, 200 mM NaCl, pH 8.0, 1% glucoside). TF concentration was determined by A₂₈₀ using molar extinction coefficient of 44410 M⁻¹ (one OD₂₈₀ is equivalent to 0.67 mg/mL). For storage, it was frozen at −70° C.

2. Preparation of crude extract: Lettuce leaf was simply homogenized with an equal volume of PBS and centrifuged at 4° C. for 5 min at 1000×g. Supernatant was used as the crude extract. For storage it was frozen and kept at −70° C.

### EXAMPLE 5

### Characterization of Purified Plant rhTF

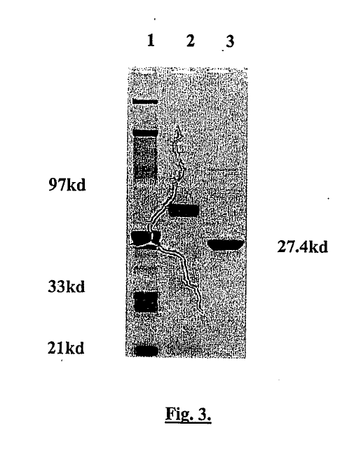

Purified lettuce TF was loaded on 12% NuPAGE Bis-Tris Gel to compare with E. coli produced rhTF₂₄₃ (FIG. 3). E. coli produced rhTF₂₄₃ molecular weight is 27.4 KD. Based on amino acid composition of lettuce TF₂₆₃, its theoretical molecular weight is 29.59 KD, however, we estimated by molecular weight markers on the gel that it's molecular mass is 35.4 KD indicating lettuce TF₂₆₃ is a glycosylated protein.

A: Prothrombin Time (PT) Assay

Lipidated rhTF and crude TF extract were diluted with PT buffer (38 mM Tris-HCl, pH 7.5, 0.1% BSA, and 14.6 mM CaCl₂) to a series dilution. PT assays were initiated by injecting 0.2 mL of lipidated rhTF or crude lettuce TF extraction into plastic twin-well cuvettes. The cuvettes each contained 0.1 mL of the plasma pre-incubated with 0.01 mL of PT buffer for 1-2 minutes. When testing anti-TF antibody effect on lettuce TF, 0.01 mL of PT buffer was replaced by cH36. An automated coagulation timer (MLA Electra 800) was used to monitor Clot time.

B: Comparative PT Assay of Purified E. coli rhTF(243aa), Purified Plant rhTF (Full Size—263 aa), and Plant rhTF Crude Extract

Purified lettuce rhTF and E. coli produced rhTF₂₄₃ (final concentration 1000 nM) were lipidated in a buffer containing 50 mM Tris-HCl, pH 7.5, 0.1% BSA, 14.6 mM CaCl₂, 0.07 mg/ml of phosphatidylcholine, and 0.03 mg/mL of phosphatidylserine at 37° C. for 30 min. Then, lipidated TF was diluted at concentrations of 3, 1, 0.5, 0.25, 0.1, 0.05 and 0.025 nM in PT assay buffer (38 mM Tris-HCl, pH 7.5, 0.1% BSA, 14.6 mM CaCl₂). Crude lettuce rhTF extract was quantified by ELISA, then diluted into the same scale as purified rhTF.

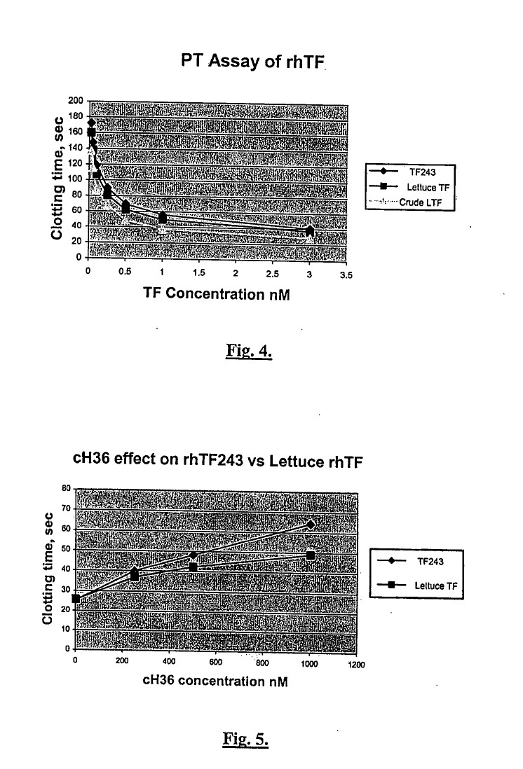

PT test was performed for these three samples. FIG. 4 shows that purified lettuce rhTF had similar clotting time as TF₂₄₃ indicating that lettuce produced rhTF is functioning, and its potency was comparable to the potency of E. coli TF₂₄₃. On the other hand, crude rhTF extract was compared with purified forms. The FIG. 4 shows that the potency of crude rhTF extract is similar as both purified forms, and pattern of dose response from three samples are same.

C: Anti-TF Monoclonal Antibody Effect on Plant rhTF

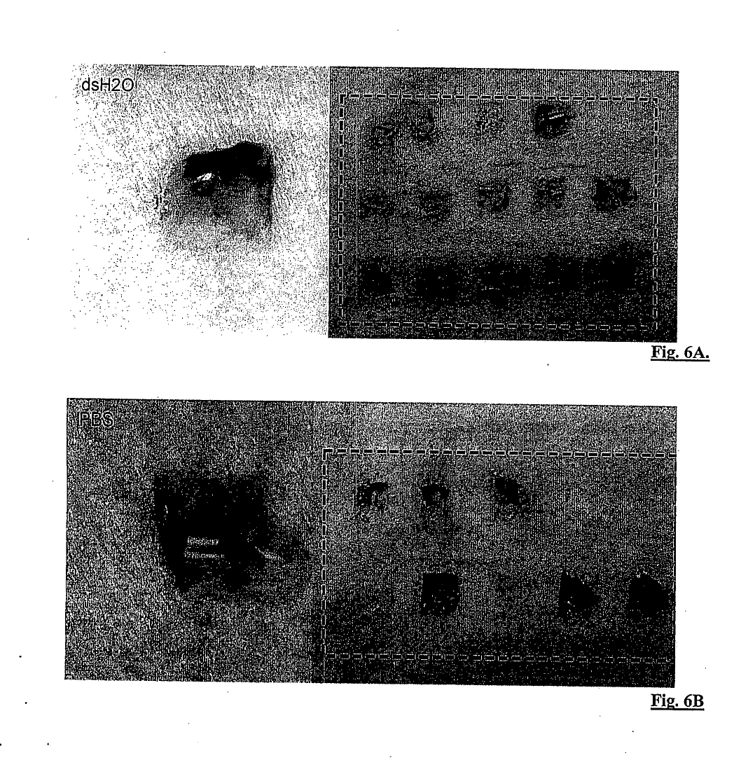

Anti-TF antibody (Sunol-cH36) effects on lettuce-produced rhTF was tested by PT assay. The antibody concentration was prepared as 0, 250 nM, 500 nM and 1000 nM. 10 μL of the antibody from each concentration was mixed and incubated with 100 μL of human plasma, respectively, then 200 μL of purified and lipidated rhTF at a concentration of 10 nM was added against different cH36 concentration. FIG. 5 indicates that anti-TF antibody, Sunol-cH36, does prolonged clotting time for lettuce-produced rhTF. For example, at antibody concentration of 500 nM, clotting time was increased from 25 sec to 42 sec, while compare to the effect to rhTF₂₄₃, which clotting time increased from 25 sec to 48 sec. Sunol-cH36, an anti-TF antibody, worked on both rhTF₂₄₃ and plant-produced rhTF₂₆₃. However, we noticed that at higher concentration of Sunol-cH36, it has less effect on lettuce-produced rhTF compared to the E. coli rhTF₂₄₃.

### EXAMPLE 6

### Effect of Plant rhTF on Hemostasis in Partial Thickness Wounds in a Porcine Model

The objective of this study was to examine the effect of a topical agent on hemostasis in partial thickness wounds in a porcine model.

A. Experimental Animal

A pig was employed as the subject in this wound model due to the morphological similarities between swine skin and human skin. One young female specific pathogen free (SPF: Ken-O-Kaw Farms, Windsor, Ill.) pig weighing ˜50 kg was used. The animal was kept in house for at least two weeks prior to initiating the experiment and was fed a basal diet ad libitum and was housed individually in an approved animal facility (meeting American Association for Accreditation of Laboratory Animal Care [AAALAC] compliance) with controlled temperature (19-21° C.) and lighting (12 hours with lighting/12 hours in darkness).

B. Wounding Technique

The flank and back of the experimental animal was clipped with standard animal clippers on the day of the experiment. The skin on both sides of each animal was prepared for wounding by washing with a non-antibiotic soap (Neutrogena Soap Bar; Johnson and Johnson, Los Angeles, Calif.) and sterile water. Each animal was anesthetized intramuscularly with tiletamine HCl plus zolazepam (1.4 mg/kg) (Telazol; Laderle Parenterals Inc, Carolina, Puerto Rico), xylazine (2.0 mg/kg) (X-jet; Phoenix Scientific Inc, St. Joseph, Mo.), and atropine (0.04 mg/kg) (Atrojet SA; Phoenix Scientific Inc, St. Joseph, Mo.) followed by mask inhalation of an isoflurane (Isothesia; Abbott Laboratories, Chicago, Ill.) and oxygen combination.

Approximately fifty-two (52) rectangular wounds measuring 10 mm×7 mm×0.4 mm deep were made in the paravertebral and thoracic area with a specialized electrokeratome fitted with a 7 mm blade. The wounds were separated from one another by approximately 15 mm of unwounded skin.

C. Treatments

Eight wounds were randomly assigned to each treatment group. Here we present results for 5 treatment groups:
1. Sterile water
2. PBS
3. E. coli rhTF₂₄₃ (100 NM)
4. Purified plant rhTF (10 nM)
5. Crude extract of plant rhTF (10 nM)

Samples were applied as a 0.2 mL volume, and were provided as materials in solution ready for application. Before experiment the specimens were placed in a 35° C. water bath and kept in the water bath until treatment. All treatments were applied within five (5) minutes of wounding, and were visually inspected for hemostasis. Evaluation of hemostasis was made by measuring the time between application of the treatment and cessation of bleeding. Each wound received its own score; the eight scores from each treatment group are averaged, and those averages are graphed (see Table 2). Representative wounds from each treatment group were photographed.

Wounds were created and blotted after commencement of punctuate bleeding. All treatments were applied immediately after wounding and blotting, and were visually inspected for hemostasis. After each wound was created, blotted and the treatment applied, the effect of the treatment was observed for time to coagulation and for the extent of bleeding at the wound site. The treatments were only applied one time.

To determine the time to coagulation, a stopwatch was started when treatment was applied and stopped when it was determined that bleeding had ceased. This time was recorded. To determine the extent of bleeding, blood accumulation at the wound site was assessed according the following scale:

TABLE 2
Hemostasis results

| | dsH₂O | | | | | | | |
|---|---|---|---|---|---|---|---|---|
| | 1 | 2 | 3 | 4 | 5 | 6 | 7 | 8 |
| time (s) | 35.00 | 42.00 | 37.00 | 40.00 | 48.00 | 42.80 | 38.28 | 40.40 |
| score | 3 | 3 | 3 | 3 | 3 | 3 | 3 | 3 |
| observations | After excess material was wiped off slow punctate | | | | | | | |
| | bleeding & mild production of clear exudate continued. | | | | | | | |
| | PBS | | | | | | | |
| | 9 | 10 | 11 | 12 | 13 | 14 | 15 | 16 |
| time (s) | 50.73 | 42.27 | 37.91 | 45.24 | 44.06 | 52.00 | 46.41 | 26.37 |
| score | 3 | 3 | 4 | 3 | 3 | 3 | 3 | 3 |
| observations | After blotting, mild punctate | | | | | | | |
| | bleeding continued | | | | | | | |
| | Ec-TF243 100 | | | | | | | |
| | 17 | 18 | 19 | 20 | 21 | 22 | 23 | 24 |
| time (s) | 22.93 | 20.18 | 16.08 | 17.45 | 15.94 | 7.91 | 8.50 | 6.67 |
| score | 2 | 2 | 2 | 2 - | 2 | 2 | 2 | 2 |
| observations | After hemostasis and blotting wounds, | | | | | | | |
| | hemostasis was permanent. | | | | | | | |
| | Pur-pTF 10 | | | | | | | |
| | 25 | 26 | 27 | 28 | 29 | 30 | 31 | 32 |
| time (s) | 17.22 | 14.45 | 15.62 | 14.50 | 12.42 | 13.44 | 17.72 | 15.42 |
| score | 2 | 2 | 2 | 1.5 | 2 | 2 | 2 | 2 |
| observations | After hemostasis and blotting wounds, | | | | | | | |
| | hemostasis was permanent. | | | | | | | |
| | Cr-pTF 10 | | | | | | | |
| | 33 | 34 | 35 | 36 | 37 | 38 | 39 | 40 |
| time (s) | 11.42 | 14.53 | 16.07 | 15.06 | 9.45 | 10.60 | 8.48 | 9.25 |
| score | 2 | 2 | 2 | 2 | 1.5 | 2 | 2 | 2 |

| | |
|---|---|
| observations | After hemostasis and blotting wounds, |
| | hemostasis was permanent. |

D. Time to Hemostasis

Wounds treated with the dsH2O and PBS controls had an average time to hemostasis of 40 and 43 seconds, respectively. Wounds treated with Ec-TF243 100, Pur-pTF10, Cr-pTF10 had an average time to hemostasis of 14, 15, and 11 seconds, respectively.

E. Score of Hemostasis

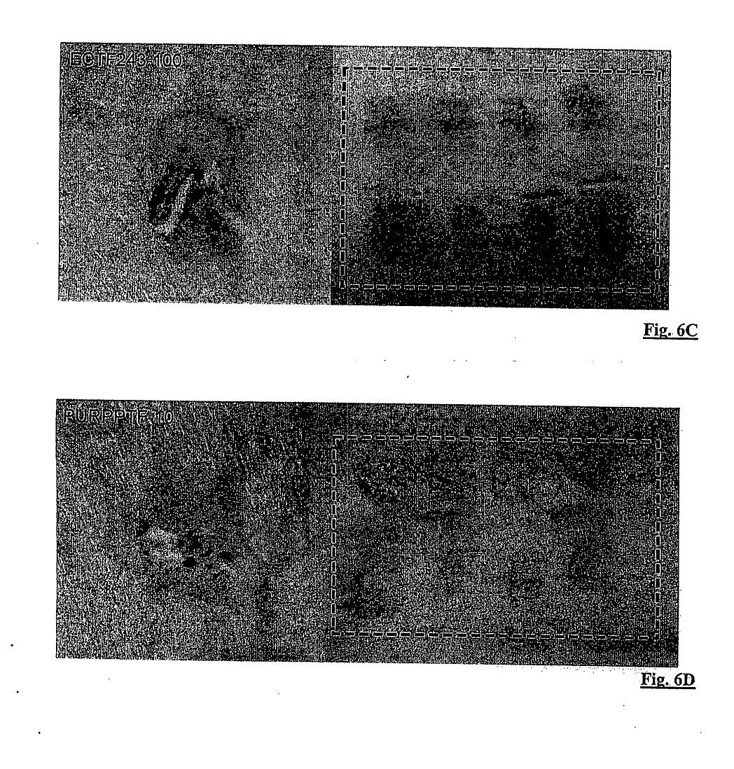

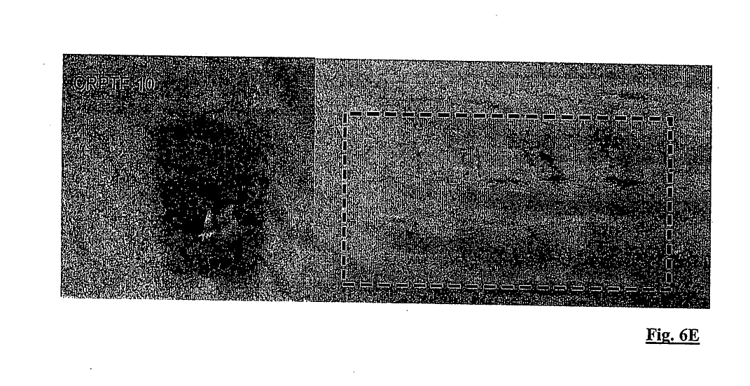

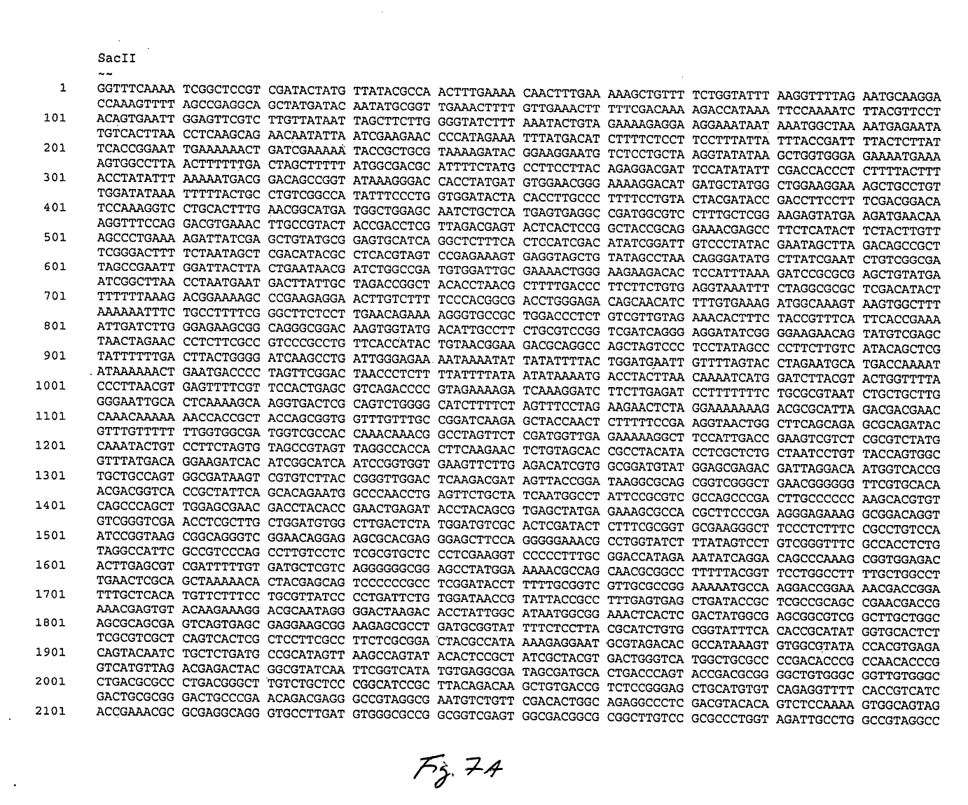

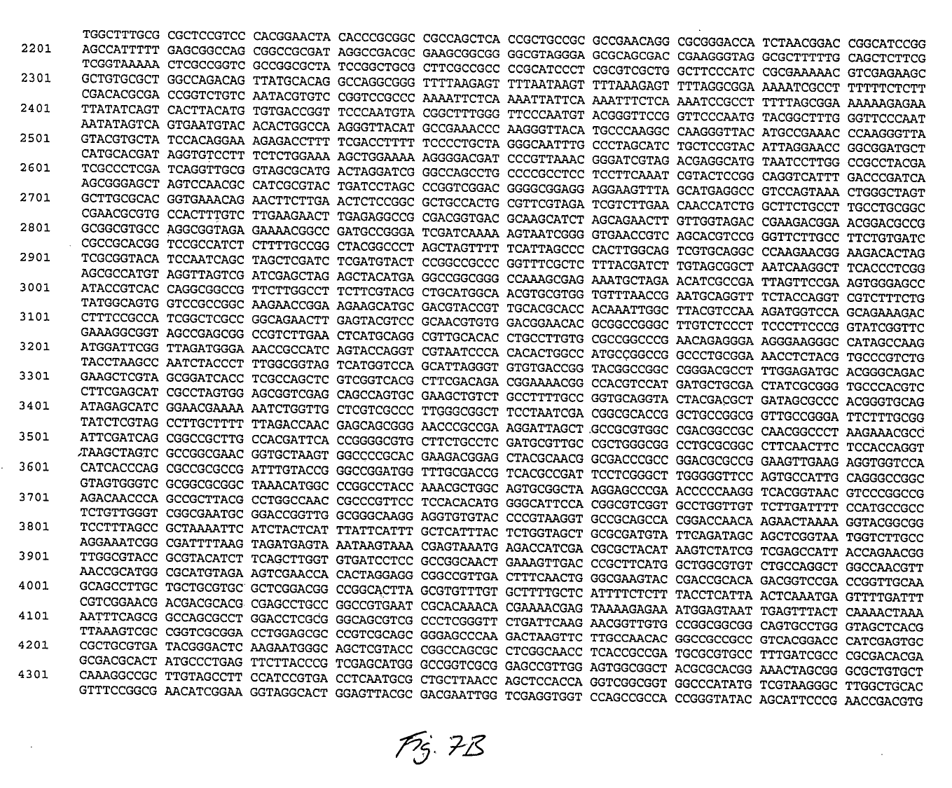

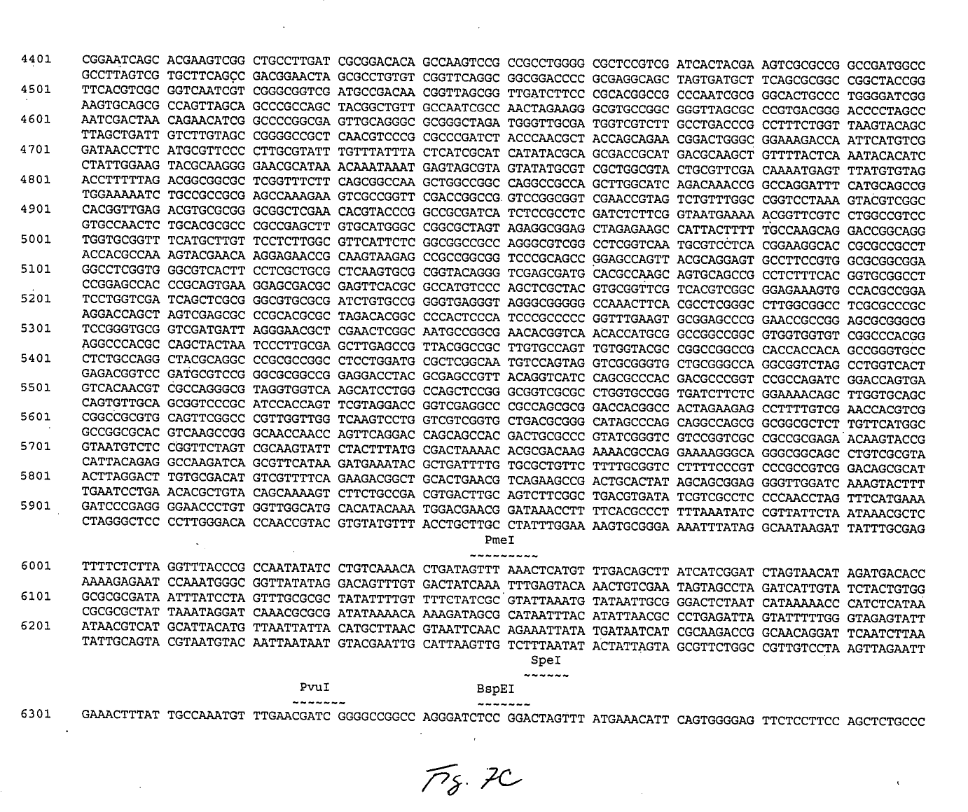

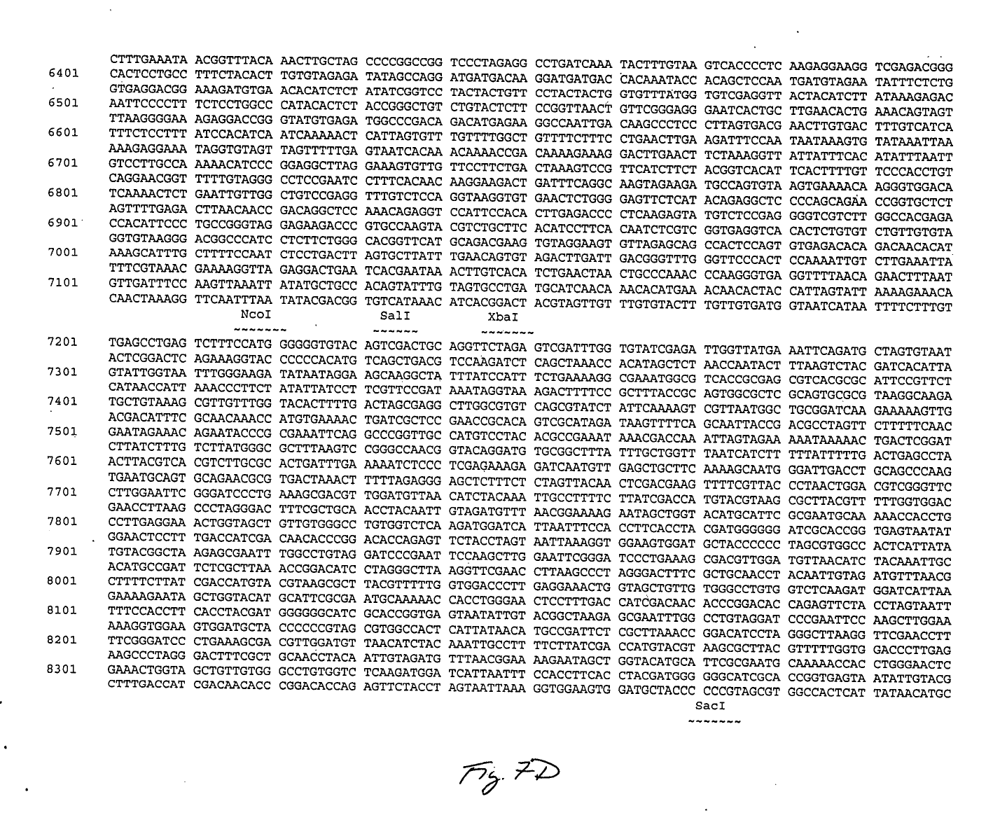

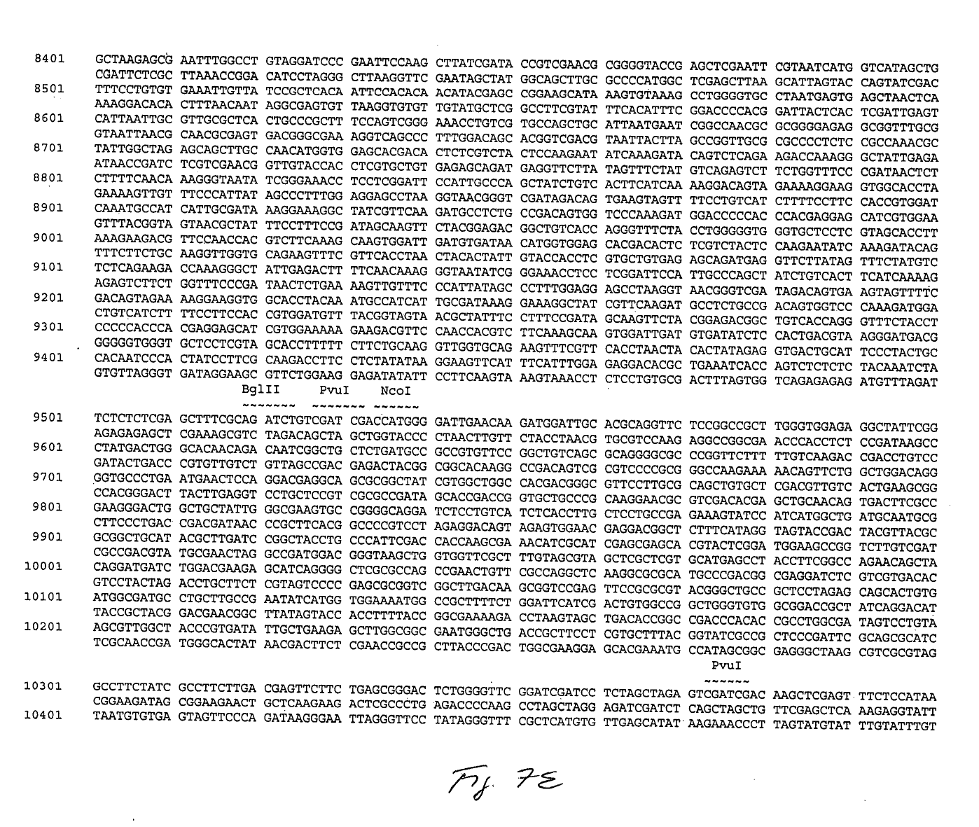

Wounds treated with the dsH₂O or PBS controls had average hemostasis scores of 3.00 and 3.12, respectively. Wounds treated with Ec-TF243 100, Pur-pTF10, or Cr-pTF10 had an average hemostasis score of 1.93, 2.00 and 1.93, respectively. Thus, Ec-TF243 100, Purp-pTF 10, and Cr-pTF 10 were able to maintain hemostasis, but both the untreated and PBS treated groups continued to bleed slowly (FIG. 6).

### EXAMPLE 7

### Stable Transgenic Plants Producing rhTF

Stable transgenic Arabidopsis thaliana (var. Columbia) plants were generated using in planta transformation by infiltration with Agrobacterium containing the pSUNP12 vector or a similar plant expression vector, pSUNP14. The pSUNP14 vector contains the rhTF gene fragment encoding amino acids 1 to 243 (i.e. terminating after the . . . A I S L H sequence shown in FIG. 1) instead for the rhTF₂₆₃ gene fragment of pSUNP12. Plants were transformed as described above, but the agro-infiltration was performed on flowering plants, a process referred to as floral dip (see for example, Clough S J and Bent A F. 1998. The Plant Journal 16:735-743). After a month and a half, the plants produced T0 seeds. Seed germination and growth on solid media containing kanamycin (Km) resulted in the selection of T0 transgenic plants. Healthy green T0 seedlings were transferred into soil to yield over 100 transgenic plants with different levels of rhTF expression. The best candidates were selected for T1 seed production. On these, line A12-9 produced 4.3 mg/kg of rhTF₂₆₃ without induction, and line A14-17 produced 2.9 mg/kg of rhTF₂₄₃ without induction. Recombinant hTF expression can be further increased through induction of the (OCS)3MAS promoter by watering plants with 1 mM salicylic acid or by treating leaves with 2,4-D as described above. Transgenic Arabidopsis plants could be induced to produce at least 20 mg/kg of rhTF by these methods. T1 seeds from these plants will be used for further screening and generation of T2 plants.

All patents and publications disclosed herein are incorporated by reference.

The invention has been described in detail with reference to preferred embodiments thereof. However, it will be appreciated that those skilled in the art, upon consideration of this disclosure, may make modification and improvements within the spirit and scope of the invention as set forth in the following claims.

1. A plant expression vector comprising:
(a) a first promoter operable in a plant,
(b) an expressible nucleic acid sequence encoding a mammalian tissue factor protein or functional fragment thereof, wherein the nucleic acid sequence is operatively associated with the promoter; and
(c) a termination sequence operatively associated with the nucleic acid sequence.

2. The plant expression vector of claim 1, wherein the vector further comprises:
(d) a second promoter operable in the plant,
(e) a selectable marker gene operatively associated with the second promoter,
(f) an Agrobacterium TR sequence; and
(g) an Agrobacterium TL sequence.

3. The plant expression vectors of claims 1 and 2, wherein the expressible nucleic acid sequence encoding the mammalian tissue factor or fragment is at least 70% identical to the mature human tissue factor sequence shown in Table I as SEQ ID NO. ______.

4. The plant expression vectors of claims 1-3, wherein the expressible nucleic acid sequence encoding the mammalian tissue factor or fragment is at least 80% identical to the mature human tissue sequence shown in Table I as SEQ ID NO. ______.

5. The plant expression vectors of claims 1-4, wherein the expressible nucleic acid sequence encoding the mammalian tissue factor or fragment is at least 90% identical to the mature human tissue sequence shown in Table I as SEQ ID NO. ______.

6. The plant expression vectors of claims 1-5, wherein the expressible nucleic acid sequence encoding the mammalian tissue factor or fragment is at least 99% identical to the mature human tissue sequence shown in Table I as SEQ ID NO. ______.

7. The plant expression vectors of claims 1-6, wherein the mammalian tissue factor or fragment encoded by the nucleic acid sequence exhibits at least 70% of the prothrombin time of the mature human tissue shown n Table I as SEQ ID NO. ______ in a standard prothrombin time (PT) assay.

8. The plant expression vectors of claims 1-7, wherein the mammalian tissue factor or fragment encoded by the nucleic acid sequence exhibits at least 80% of the prothrombin time of the mature human tissue factor protein shown in Table I as SEQ ID NO. ______ in a standard prothrombin time (PT) assay.

9. The plant expression vectors of claims 1-8, wherein the mammalian tissue factor or fragment encoded by the nucleic acid sequence exhibits at least 90% of the prothrombin time of the mature human tissue factor protein shown in Table I as SEQ ID NO. ______ in a standard prothrombin time (PT) assay.

10. The plant expression vectors of claims 1-9, wherein the mammalian tissue factor or fragment encoded by the nucleic acid sequence exhibits at least 99% of the prothrombin time of the mature human tissue factor protein shown in Table I as SEQ ID NO. ______ in a standard prothrombin time (PT) assay.

11. The plant expression vector of claims 1-10, wherein the first promoter is inducible in the plant.

12. The plant expression vector of claims 1-10, wherein the first promoter is constitutive in the plant.

13. The plant expression vector of claims 1-12, wherein the nucleic acid sequence encoding the tissue factor further comprises sequence encoding a plant- or mammal-derived secretion signal peptide.

14. The plant expression vector of claim 13, wherein the plant-derived signal sequence is from tomato, rice, tobacco, mustard, cotton, maize or alfalfa.

15. The plant expression vector of claims 1-14, wherein components (a), and (d)-(g) are positioned on the vector in a 5′ to 3′ direction.

16. The plant expression vector of claim 15, wherein components (b) and (c) are positioned in a reverse orientation with respect to the positions of components (a), and (d)-(f) on the vector.

17. A transgenic plant comprising transformed plant cells in which the cells comprise the replicable expression vector of any one of claims 1-16.

18. The transgenic plant of claim 17, wherein the plant is a dicot.

19. The transgenic plant of claim 18, wherein the plant is a monocot.

20. The transgenic plant of claim 17, wherein the plant is a member of the family Fabaceae, Solanaceae, Brassicaceae, Rosaceae or Compositae.

21. The transgenic plant of claim 20, wherein the plant is duckweed, tobacco (Nicotiana tabacum), mustard (Arabidopsis), potato (Solanum tuberosum), soybean (glycine max), tomato (Lycopersicon esculentum), cassaya (Manihot esculenta), beets, peanuts (Arachis hypogaea), cotton (Gossypium hirsutum), citrus trees (Citrus spp.), corn or maize (Zea mays), beans (e.g., green beans (Phaseolus vulgaris) and lima beans (Phaseolus limensis)), peas (Lathyrus spp.), sugarbeet, sunflower, carrot, celery, flax, cabbage (and other cruciferious plants), pepper, mango, peach, apple, pear, banana, sunflower, onion, strawberry, alfalfa, oat, wheat, rye, rice, barley, sorghum and canola.

22. The transgenic plant of claim 20, wherein the plant is of the genus Lactucua.

23. The transgenic plant of claim 22, wherein the plant is L. sativa (lettuce).

24. The transgenic plant of claim 17, wherein the plant is a cereal.

24a. The transgenic plant of claim 17 wherein glycosylation has been genetically altered.

25. A progeny plant, cutting, slip, cultivar, or seed from the transgenic plant of claims 17-24, wherein the progeny plant, cutting, slip, cultivar, or seed comprises the expressible nucleic acid sequence encoding the mammalian tissue factor protein or functional fragment thereof.

26. A transgenic plant obtained from the cutting, slip, cultivar, or seed of claim 25, wherein the transgenic plant comprises the expressible nucleic acid sequence encoding the mammalian tissue factor protein or functional fragment thereof.

27. A transgenic lettuce (L. sativa) comprising transformed plant cells in which the cells comprise:
(a) a first promoter operable in a plant,
(b) an expressible nucleic acid sequence encoding a mammalian tissue factor protein or functional fragment thereof, wherein the nucleic acid sequence is operatively associated with the promoter; and
(c) a termination sequence operatively associated with the nucleic acid sequence.
(d) a second promoter operable in the plant,
(e) a selectable marker gene operatively associated with the second promoter,
(f) an Agrobacterium TR sequence, and
(g) an Agrobacterium TL sequence;
wherein the expressible nucleic acid sequence encoding the mammalian tissue factor protein or fragment further encodes a plant specific signal peptide operatively linked to the tissue factor protein or fragment.

28. The transgenic lettuce (L. sativa) of claim 27, wherein,
i) the first promoter is inducible or constitutive,
ii) the expressible nucleic acid sequence encodes a mature human tissue factor protein as represented by the sequence shown in Table I as SEQ ID NO. ______, a functional fragment thereof or a sequence at least about 70% identical to mature human tissue factor protein shown in Table I as SEQ ID NO. ______nd exhibiting at least 70% of the prothrombin time of the mature human tissue factor protein shown in SEQ ID NO. ______,
iii) the termination sequence is Nos-3′,
iv) the second promoter is a 35S promoter; and
v) the selectable marker gene is NPTII.

29. A recombinant mammalian tissue factor or a functional fragment thereof obtained from the transgenic plant of claims 1-28.

30. A recombinant mammalian tissue factor or functional fragment thereof comprising an operatively linked plant-derived signal peptide.

31. The recombinant mammalian tissue factor of claim 30, wherein the signal sequence is derived from tomato, rice, tobacco, mustard, cotton, maize, or alafalfa.

32. The recombinant mammalian tissue factor of claims 30 and 31, wherein the tissue factor sequence is represented by FIG. 1B (SEQ ID NO. ______); or a sequence at least about 70% identical to that SEQ ID NO. ______.

33. The recombinant mammalian tissue factor of claim 32, wherein the tissue factor sequence exhibits at least 70% of the prothrombin time of the mature human tissue factor protein shown in Table I as SEQ ID NO.

34. A recombinant mammalian tissue factor or functional fragment thereof comprising at least one plant-specific glycan.

35. The recombinant mammalian tissue factor of claim 34, wherein the plant-specific glycan is an α(1,3) fucose group.

36. The recombinant mammalian tissue factor of claim 34, wherein the plant-specific glycan is an β(1,2) xylose group.

37. The recombinant mammalian tissue factor of claim 34, wherein the plant-specific glycan is an α(1,3) fucose and a β(1,2) xylose group.

38. The recombinant mammalian tissue factor of claims 35-37, wherein the α(1,3) fucose is covalently linked to a N-acetylglucosamine (GlcNAc) group.

39. The recombinant mammalian tissue factor of claims 35-37, wherein the β(1,2) xylose group is covalently linked to a mannose group.

40. The recombinant mammalian tissue factor of claims 29-39, wherein the tissue factor sequence has sequence as represented by FIG. 1B (SEQ ID NO.); ______); or a sequence at least about 70% identical to that SEQ ID NO. ______.

41. The recombinant mammalian tissue factor of claim 40, wherein the tissue factor sequence exhibits at least 70% of the prothrombin time of the mature human tissue factor protein shown in Table I as SEQ ID NO.

42. A substantially purified preparation of the recombinant mammalian tissue factor of claims 29-41.

41. A substantially purified preparation of the recombinant mammalian tissue factor of claims 29-41 and at least one of phosphatidylcholine and phosphatidylserine.

42. A crude extract comprising recombinant tissue factor (rhTF) or a functional fragment thereof suspended in an aqueous fraction obtained from the transgenic plant of claims 17-28.

43. The crude extract of claim 42, wherein the recombinant tissue factor or fragment thereof further comprises operatively linked plant- or mammalian-derived signal peptide.

44. The crude extract of claims 42-43 further comprising the recombinant tissue factor or fragment thereof, the tissue factor or fragment further comprising an plant-specific glycan.

45. The crude extract of claims 42-44, wherein the extract further comprises a pharmaceutically acceptable solution.

46. The crude extract of claim 45, wherein the solution is water, phosphate buffered saline (PBS) or Tris-buffered saline.

47. An anticoagulant comprising at least one of the compositions of claims 29-46.

48. The anticoagulant of claim 47 further comprising at least one of a pharmaceutically acceptable vehicle, additive, or stabilizer.

49. A method of making the transgenic plant of claims 17-28, the method comprising:
i) administering to a suitable plant or plant tissue at least one replicable expression vector comprising (a) a first promoter operable in a plant, (b) an expressible nucleic acid sequence encoding a mammalian tissue factor protein or functional fragment thereof, wherein the nucleic acid sequence is operatively associated with the promoter; and (c) a termination sequence operatively associated with the nucleic acid sequence; and
ii) incubating the plant or plant tissue under conditions conducive to expressing the mammalian tissue factor protein or functional fragment thereof, thereby making the transgenic plant.

50. A method of making the transgenic plant of claims 17-28 comprising:
i) administering to a suitable Agrobacterium strain at least one replicable expression vector comprising (a) a first promoter operable in a plant, (b) an expressible nucleic acid sequence encoding a mammalian tissue factor protein or functional fragment thereof, wherein the nucleic acid sequence is operatively associated with the promoter; and (c) a termination sequence operatively associated with the nucleic acid sequence; and
ii) incubating the bacterium with a suitable plant or plant tissue under conditions conducive to transforming the plant or plant tissue with the vector to make the transgenic plant.

51. A method of making the isolated recombinant tissue factor or functional fragment thereof of claims 29-41, the method comprising:
i) administering to a suitable plant or plant tissue at least one replicable expression vector comprising (a) a first promoter operable in a plant, (b) an expressible nucleic acid sequence encoding a mammalian tissue factor protein or functional fragment thereof, wherein the nucleic acid sequence is operatively associated with the promoter; and (c) a termination sequence operatively associated with the nucleic acid sequence; and
ii) incubating the plant or plant tissue under conditions conducive to expressing the mammalian tissue factor protein or functional fragment thereof, thereby making the transgenic plant; and
iii) preparing a crude extract from the transgenic plant; and
iv) isolating the recombinant tissue factor or functional fragment thereof from the crude extract.

52. A method of making the isolated recombinant tissue factor or functional fragment thereof of claims 29-41, the method comprising:
i) administering to a suitable Agrobacterium strain at least one replicable expression vector comprising (a) a first promoter operable in a plant, (b) an expressible nucleic acid sequence encoding a mammalian tissue factor protein or functional fragment thereof, wherein the nucleic acid sequence is operatively associated with the promoter; and (c) a termination sequence operatively associated with the nucleic acid sequence; and
ii) incubating the bacterium with a suitable plant or plant tissue under conditions conducive to transforming the plant or plant tissue with the vector to make a transgenic plant; and
iii) preparing a crude extract from the transgenic plant; and
iv) isolating the recombinant tissue factor or functional fragment thereof from the crude extract.

53. The method of claim 51 or 52, wherein the method further comprises purifying the recombinant tissue factor or functional fragment thereof by at least one of: chromatography, centrifugation, or immunoprecipitation.

54. The method of claim 53, wherein the resulting recombinant tissue factor or fragment thereof is substantially pure.

55. A method for preventing or treating blood loss in a subject, the method comprising contacting the subject with a therapeutically effective amount of the anticoagulant of claims 47 or 48.

56. The method of claim 55, wherein the subject is a human patient in need of treatment.

56a. The method of claim 55, wherein the subject is a mammal in need of treatment.

57. An absorbent comprising the anticoagulant of claims 47 or 48.

58. The absorbent of claim 57, wherein the adsorbent is medical gauze.

59. The absorbent of claim 58, wherein the medical gauze comprises cotton fibers or a fiber blend comprising cotton.

60. The absorbent of claims 57-59, wherein the adsorbent comprises a pharmaceutically acceptable solution.

61. An adhesive support comprising the absorbent of claims 57-60.

62. The adhesive support of claim 61, wherein the adhesive support is sterile.

63. A wrapper container comprising the adhesive support of claim 62, wherein the wrapper container is essentially impervious to water and air.

64. The replicable plant expression vector of claims 1-11, wherein the vector further comprises sequence that suppresses gene silencing.

65. The transgenic lettuce (L. sativa) of claim 28, wherein the first promoter is a 2,4,-D inducible (OCS)3Mas promoter.

66. The recombinant mammalian tissue factor of claims 34-39, wherein the plant-specific glycan of the tissue factor lacks a terminal galactose group.

67. The recombinant mammalian tissue factor of claim 66, wherein the plant-specific glycan of the tissue factor further lacks a terminal acetylneuraminic acid (NeuAc; sialic acid) group.

68. The recombinant tissue factor of claim 67, wherein the sialic-acid group is covalently linked to the galactose group.

69. The method of claim 55, wherein the anticoagulant is delivered to the subject by one or more of pouring or spaying the anticoagulant on or near a wound.

70. A method for promoting wound healing in a subject, the method comprising contacting the subject with a therapeutically effective amount of the anticoagulant of claims 47 or 48.

71. A method for promoting angiogenesis in a subject, the method comprising contacting the subject with a therapeutically effective amount of the anticoagulant of claims 47 or 48.

72. A method for promoting vascular remodeling in a subject, the method comprising contacting the subject with a therapeutically effective amount of the anticoagulant of claims 47 or 48.
